SEQUENCE LISTING (30 sequences)

SEQ ID NO: 1 - DNA, 1828 bp, Homo sapiens
  CDS (527)..(1825)
ccacgacaacccactgcaagcagatccagcagctgcttcctgtaatgcaggactccccaggagcacccaggcatgggcagcaacccctgcatgccattctttttgggctcccgagaacagggagataaacaccaccatcatctgagagccgggaaggggaaggcgagggtgtgtaggcggcacgaatgctccgttgagagacgcggctttcggcaagaactggattcgtggcgccacaagctcattcactgtgtaggtcccgtttccctctgtgcggcggccggcgggaccataagggcttaactcatatatttaacccccctccaaaaaggtttgaaagtattcttgaagggctgtttggacctgcattattaaaagatctcagtttatttaaagactgtgaacctgaaagcatttctgattggacttttgatgaaaactgtttattctgttgcttgagaagagataaagtaaagacagtccctgggtctccgaccccaatattcccctagtggcccgtgagatcatgcagcgaatgatccaacaatttgctgctgaatatacctcaaaaaatagctctactcaggaccccagccagcccaatagcacaaagaaccaaagcctgccgaaagcatctccagtcaccacctctcccacggctgcaactactcagaaccctgtgctcagcaaacttctcatggctgaccaagactcacctctggaccttactgtcagaaagtctcagtcagaacctagcgaacaagacggtgtacttgatctgtccactaagaaaagtccatgtgctggcagcacttccctgagccactctccaggctgctccagtactcaagggaacgggcgacctgggagacccagccagtaccgcccagacggacttcggagtggtgatggggtacctccaagaagcttacaggatggaaccagggaaggttttggacactccacatcactcaaagttccactggctcgatccctgcagattagtgaagaactactgagcagaaaccaattgtccacagctgccagccttgggccatctggattacagaatcatggacaacacttaatattatccagggaagcctcttgggcaaaaccacattacgagttcaacctcagccgtatgaagttcaggggaaatggtgcactcagcaacatcagtgaccttccttttcttgcagaaaactctgcctttccaaaaatggcacttcaagcaaaacaagatggaaaaaaggacgtgagccattcatctcctgtagatttaaagataccacaagttcgaggaatggatctttcttgggagtctcgcactggtgatcagtacagctatagctctttggtaatgggttcacaaacggagagcgcgcttagtaaaaaattaagggctattcttccaaaacaaagtagaaaaagcatgttagatgctgggcccgattcttggggctcagatgctgagcagtctacccctggacagccatatcccacatcggatcaagaaggagaccctggctccaagcagcctcggaagaaaagagggcgttacagacagtacaacagtgagatactggaggaagcaatctcagtggttatgagtggaaaaatgagtgtttccaaagctcagagtatttatgggattccccacagtacactggagtacaaagtaaaggagaggctgggcactttgaaaaaccctccaaagaaaaagatgaaattaatgaggtcggaggggccagatgtttctgtaaagattgaattagatccccagggagaggcagcacaaagtgcaaatgaatcaaaaaacgagtag

SEQ ID NO: 2 - protein, 433 aa, Homo sapiens
MQRMIQQFAAEYTSKNSSTQDPSQPNSTKNQSLPKASPVTTSPTAATTQNPVLSKLLMADQDSPLDLTVRKSQSEPSEQDGVLDLSTKKSPCAGSTSLSHSPGCSSTQGNGRPGRPSQYRPDGLRSGDGVPPRSLQDGTREGFGHSTSLKVPLARSLQISEELLSRNQLSTAASLGPSGLQNHGQHLILSREASWAKPHYEFNLSRMKFRGNGALSNISDLPFLAENSAFPKMALQAKQDGKKDVSHSSPVDLKIPQVRGMDLSWESRTGDQYSYSSLVMGSQTESALSKKLRAILPKQSRKSMLDAGPDSWGSDAEQSTPGQPYPTSDQEGDPGSKQPRKKRGRYRQYNSEILEEAISVVMSGKMSVSKAQSIYGIPHSTLEYKVKERLGTLKNPPKKKMKLMRSEGPDVSVKIELDPQGEAAQSANESKNE

SEQ ID NO: 3 - protein, 60 aa, Homo sapiens
KQPKKKRGRYRQYNSEILEEAISVVMSGKMSVSKAQSIYGIPHSTLEYKVKERLGTLKNP

SEQ ID NO: 4 - protein, 17 aa, Artificial Sequence
  Description of Artificial Sequence Synthetic peptide
QDPSQPNSTKNQSLPKA

SEQ ID NO: 5 - DNA, 30 bp, Artificial Sequence
  Description of Artificial Sequence Synthetic primer
ccggaattccggatgaccatgaccctccac

SEQ ID NO: 6 - DNA, 31 bp, Artificial Sequence
  Description of Artificial Sequence Synthetic primer
cgggatcccgtcaaaggtggacctgatcatg

SEQ ID NO: 7 - DNA, 40 bp, Artificial Sequence
  Description of Artificial Sequence Synthetic primer
ccggaattccggcccgggcatgagacagtccctgggtctc

SEQ ID NO: 8 - DNA, 20 bp, Artificial Sequence
  Description of Artificial Sequence Synthetic primer
ttcttggaggtaccccatca

SEQ ID NO: 9 - DNA, 30 bp, Artificial Sequence
  Description of Artificial Sequence Synthetic primer
cgcggatccgcgatgcagcgaatgatccaa

SEQ ID NO: 10 - DNA, 29 bp, Artificial Sequence
  Description of Artificial Sequence Synthetic primer
ggaattccctactcgttttttgattcatt

SEQ ID NO: 11 - DNA, 31 bp, Artificial Sequence
  Description of Artificial Sequence Synthetic primer
ctagctagccaccatgcagcgaatgatccaa

SEQ ID NO: 12 - DNA, 29 bp, Artificial Sequence
  Description of Artificial Sequence Synthetic primer
ctagctagccgctcgttttttgattcatt

SEQ ID NO: 13 - DNA, 26 bp, Artificial Sequence
  Description of Artificial Sequence Synthetic primer
cggaattccagcgaatgatccaacaa

SEQ ID NO: 14 - DNA, 33 bp, Artificial Sequence
  Description of Artificial Sequence Synthetic primer
cgcggatccgcgctactcgttttttgattcatt

SEQ ID NO: 15 - protein, 7 aa, Artificial Sequence
  Description of Artificial Sequence Synthetic peptide motif sequence
PLDLTVR

SEQ ID NO: 16 - protein, 7 aa, Artificial Sequence
  Description of Artificial Sequence Synthetic peptide motif sequence
VLDLSTK

SEQ ID NO: 17 - protein, 5 aa, Artificial Sequence
  Description of Artificial Sequence Synthetic peptide
LSKAA

SEQ ID NO: 18 - protein, 9 aa, Artificial Sequence
  Description of Artificial Sequence Synthetic peptide
  MOD_RES (2): variable amino acid
  MOD_RES (4): variable amino acid
  MOD_RES (6): variable amino acid
  MOD_RES (8): variable amino acid
LXLXLXIXL

SEQ ID NO: 19 - protein, 5 aa, Artificial Sequence
  Description of Artificial Sequence Synthetic peptide
LSKLL

SEQ ID NO: 20 - protein, 7 aa, Artificial Sequence
  Description of Artificial Sequence Synthetic peptide motif sequence
PRKKRGR

SEQ ID NO: 21 - protein, 7 aa, Artificial Sequence
  Description of Artificial Sequence Synthetic peptide motif sequence
  MOD_RES (5): Ser or Thr
  MOD_RES (6): variable amino acid
  MOD_RES (7): Arg or Lys
PLDLXXX

SEQ ID NO: 22 - protein, 8 aa, Artificial Sequence
  Description of Artificial Sequence Synthetic peptide motif sequence
  MOD_RES (2): variable amino acid
  MOD_RES (6)..(7): variable amino acid
PXLDLXXR

SEQ ID NO: 23 - protein, 60 aa, Drosophila melanogaster
EGIELSRSNPTPWSEDAMNEALNSVRLGQMSINQAAIHYNLPYSSLYGRFKRGKYDVVAN

SEQ ID NO: 24 - protein, 60 aa, Drosophila melanogaster
KGTRPKRCKYRNYDRDSLVEAVKAVQRGEMSVHRAGSYYGVPHSTLEYKVKERHLMRPRK

SEQ ID NO: 25 - protein, 60 aa, Caenorhabditis elegans
KRSRPKRGQYRKYDKNALDEAVRSVRRGEMTVHRAGSFFGVPHSTLEYKVKERNLMRKKK

SEQ ID NO: 26 - protein, 60 aa, Apis mellifera
KGTRPKRGKYRNYDRDSLVEAVRAVQRGEMSVHRAGSYYGVPHSTLFYKVKERHLMRPRK

SEQ ID NO: 27 - protein, 60 aa, Apis mellifera
VPVVGAGGGRRAYTEEELQAALRDIQSGKLGTRRAAVIYGIPRSTLRNKVYKLAMERERD

SEQ ID NO: 28 - protein, 60 aa, Drosophila melanogaster
SGEKGGFNGPKAWTQDDMNSALDALKNQNMSLTKASAIYGIPSTTLWQRAHRMGIETPKK

SEQ ID NO: 29 - protein, 60 aa, Drosophila melanogaster
IETPKKEGGTKSWNEDALQNALEALRSGQISANKASKAFGIPSSTLYKIARREGIRLAAP

SEQ ID NO: 30 - protein, 60 aa, Drosophila melanogaster
RLAAPFNAAPTTWTPEDLERALEAIRAGNTSVQKASAEFGIPTGTLYGRCKREGIELSRS

(19) United States
(12) Patent Application Publication
(10) Pub. No.: US 2007/0054353 A1
(43) Pub. Date: Mar. 8, 2007

(54) Nuclear receptor transcriptional corepressor and uses thereof

(76) Inventors: John White, Montreal (CA); Isabelle Fernandes, Livry (FR)

Correspondence Address: KEOWN & ASSOCIATES, 500 WEST CUMMINGS PARK, SUITE 1200, WOBURN, MA, 01801, US

(21) Appl. No.: 10/529,512

(22) PCT Filed: Sep. 25, 2003
(86) PCT No.: PCT/CA03/01477

§ 371 Date: Dec. 23, 2005

Related U.S. Application Data
(60) Provisional application No. 60/413,602, filed on Sep. 26, 2002.

(51) Int. Cl. C12P 21/06; C12N 9/99; C07H 21/04; C07K 14/575

(52) U.S. Cl. 435/69.1; 435/320.1; 435/325; 435/184; 530/399; 536/23.2

(57) ABSTRACT

The present invention provides a novel class of transcriptional corepressor polypeptides having an amino acid sequence which comprises at least one LXXLL nuclear receptor interacting NR box motif wherein L is leucine and X is any amino acid residue, and which are operably interactable with a nuclear receptor to actively repress transcription of DNA. The corepressor is widely expressed in fetal and adult tissues and attenuates agonist-activated nuclear receptor signaling by multiple mechanisms. Also provided are methods and uses for the novel class of transcriptional corepressors to repressing transcription in a cell.

### BACKGROUND OF THE INVENTION

a) Field of the Invention

This invention relates generally to corepressor polypeptides and uses thereof, more particularly, a novel class of corepressor polypeptides having an amino acid sequence which comprises at least one LXXLL NR box motif, corepressor polypeptides having within their amino acid sequence at least two C-terminal binding protein interaction motifs, variants of the corepressor polypeptides, polynucleotides encoding for the corepressor polypeptides, expression vectors comprising the polynucleotides, host cells stably transformed with the expression vectors, antibodies that bind to the polypeptide corepressors, transgenic knock-out mice having disruption in an endogenous gene which encodes for the corepressor polypeptides, methods of modulating a cell, methods of inhibiting ligand-dependent transactivation in a cell, methods of repressing nuclear-receptor mediated transcription in a cell, methods of modulating steroid hormone signaling in a cell, methods of regulating gene expression, methods for assaying for compounds capable of modulating the activity of the corepressor polypeptides, and methods for assaying for compounds capable of affording selective recruitment of the corepressor polypeptides.

b) Brief Description of the Prior Art

Nuclear receptors are ligand-regulated transcription factors whose activities are controlled by a range of lipophilic extracellular signals. They directly regulate transcription of genes whose products control many aspects of physiology and metabolism (Chawla, A. et al. (2001) Science, 294, 1866-70). Different receptors have distinct ligand binding, DNA binding and transcriptional regulation properties (Chawla, A. et al. (2001) Science, 294, 1866-70).

Receptors are composed of a series of conserved domains, A-F. N-terminal A/B regions contain transactivating domains (activating function-1; AF-1), which can cooperate with AF-2, located in the C-terminal ligand-binding domain (LBD). Crystal structures of agonist- and antagonist-bound LBDs have revealed highly conserved α helical structures (Brzozowski, A. M. et al. (1997) Nature, 389, 753-8). Agonist binding induces conformational changes that reorient the C-terminal AF-2 helix (helix 12) to create a binding pocket recognized by coactivators.

Several coregulatory proteins control nuclear receptor function (Rosenfeld M. G. and Glass, C. K. (2001) J. Biol. Chem., 276, 36865-68). Their diversity suggests that transcriptional activation by receptors occurs through recruitment of multiple factors acting sequentially or combinatorially. Coactivation of the p160 family, SRC1/NCoA1, TIF-2/GRIP-1 and pCIP/AIB1/RAC3/ACTR/TRAM-1, which interact with ligand-bound receptors through LXXLL motifs (wherein L is leucine and X is any amino acid), known as NR boxes. Co-crystallographic studies of ligand-bound nuclear receptors revealed α-helical NR boxes oriented within a hydrophobic pocket containing the repositioned helix 12 by a charge clamp formed by conserved lysine and glutamate residues in helices 3 and 12, respectively (Shiau, A. K. et al. (1998) Cell, 95, 927-37). P160 coactivators recruit other proteins essential for transactivation, including CREB binding protein (CBP) and its homologue. Several coactivators including CBP/p300 and associated factor p/CAF possess histone acetyltransferase activity, required for chromatin remodeling and subsequent access of the transcriptional machinery to promoters.

Corepressors NCoR and SMRT mediate ligand-independent repression by thyroid and retinoic acid receptors and recruit multi-protein complexes implicated in transcriptional repression and histone deacetylation. Histone deacetylases (HDACs) identified to date fall into three classes based on homology, domain structure, subcellular localization, and catalytic properties (Khochbin, S. et al. (2001) Curr. Opinion Genet Dev. 11, 162-6). NCoR and SMRT are components of several different complexes containing distinct combinations of ancillary proteins and class I or class II HDACs (Rosenfeld M. G. and Glass, C. K. (2001) J. Biol. Chem., 276, 36865-68), suggesting that their function depends on cell type, combinations of transcription factors bound to specific promoters, and phase of the cell cycle.

There exists a need in the art for identification of novel corepressor polypeptides that serve as transcriptional corepressors. The present invention fulfills these and other needs in the art.

### SUMMARY OF THE INVENTION

In accordance with the present invention, there is provided novel corepressor polypeptides, polynucleotides encoding the corepressor polypeptide, and uses thereof.

More particularly, the present invention reduces the difficulties and disadvantages of the prior art by providing novel corepressor polypeptides that can interact with nuclear receptors such as ERα, through a single NR box motif. This is unlike known corepressors, NCoR and SMRT. The novel corepresor polypeptides of the present invention are expressed from the earliest stages of mammalian development and are operable to couple specific class I and class II HDACs to ligand-bound nuclear receptors. Corepressor polypeptides of the present invention represent a novel class of nuclear receptor corepressor that acts to attenuate signaling by ligand-bound receptors. Corepressor polypeptides of the present invention can interact with agonist-bound nuclear receptors in a ligand or partially ligand-dependent manner through an NR box. Moreover, corepressor polypeptides of the present invention represent a new class of corepressor that can couple specific HDACs to ligand-activated nuclear receptors and attenuate their signaling.

Therefore in a first embodiment of the present invention, there is provided an isolated corepressor polypeptide having an amino acid sequence which comprises at least one LXXLL nuclear receptor interacting NR box motif wherein L is leucine and X is any amino acid residue, said polypeptide operably interactable with a nuclear receptor to actively repress transcription of DNA.

In another aspect of the present invention, there is provided an isolated polypeptide encoded by the nucleotide sequence at set forth in FIG. 1D (SEQ ID NO:1).

In another aspect of the present invention, there is provided an isolated corepressor polypeptide essentially having an amino acid sequence as set forth at FIG. 1D (SEQ ID NO:2) comprising at least one modification of the amino acid sequence.

In another aspect of the present invention, there is provided an isolated corepressor polypeptide having within its amino acid sequence at least two C-terminal binding protein interaction motifs, the first C-terminal binding protein interaction motif comprising the sequence PLDLTVR, and the second C-terminal binding protein interaction motif comprising the sequence VLDLSTK. The corepressor polypeptide is operably interactable with a C-terminal binding protein (CtBP) corepressor in a pathway to repress expression of DNA. In one embodiment, the isolated polypeptide comprises the amino acid sequence as set forth in FIG. 1D (SEQ ID NO:2).

In yet another aspect of the present invention, there is provided an isolated polynucleotide coding for a corepressor polypeptide of the present invention.

In yet another aspect of the present invention, there is provided an expression vector comprising a corepressor polynucleotide of the present invention operably linked to a promoter for expression in a host cell.

In yet another aspect of the present invention, there is provided a host cell stably transformed with an expression vector of the present invention.

In yet another aspect of the present invention, there is provided an antibody that binds to a corepressor polypeptide of the present invention.

In yet another aspect of the present invention, there is provided a transgenic knock-out mouse having disruption in an endogenous gene which encodes for a corepressor polypeptide of the present invention. The disruption is introduced into its genome by a recombinant DNA construct stably integrated into the genome of the mouse or an ancestor thereof, wherein the disruption of the corepressor gene reduces expression of the corepressor causing altered active transcription of DNA associated with the corepressor.

In yet another aspect of the present invention, there is provided a method of modulating a cell having a gene which encodes for a corepressor polypeptide of the present invention, comprising the steps of introducing into the cell an isolated polynucleotide having essentially the amino acid sequence as set forth in FIG. 1D (SEQ ID NO:2) with at least one modification in the amino acid sequence, whereby expression of the corepressor polypeptide is modulated.

In yet another aspect of the present invention, there is provided a method of inhibiting ligand-dependent transactivation in a cell by one of a class I and class II nuclear receptor comprising subjecting the cell to a corepressor amount of a polypeptide of the present invention. In a preferred embodiment, the nuclear receptor comprises a member of the nuclear receptor superfamily. In another preferred embodiment, the nuclear receptor is selected from the group consisting of ERα, ERβ, GR, PR, VDR, RARα, RARβ, and RARγ.

In yet another aspect of the present invention, there is provided a method of repressing nuclear-receptor mediated transcription in a cell comprising providing a ligand-dependent corepressor amount of a corepressor polypeptide of the present invention to the cell.

In yet another aspect of the present invention, there is provided a method of modulating steroid hormone signaling in a cell comprising providing a ligand-dependent corepressor amount of a polypeptide of the present invention to the cell.

In yet another aspect of the present invention, there is provided a method of regulating gene expression in a cell comprising providing a corepressor polypeptide of the present invention, wherein the polypeptide is operable to interact with at least one protein in a pathway to regulate gene expression.

In yet another aspect of the present invention, there is provided a use of a corepressor polypeptide of the present invention to inhibit ligand-dependent transactivation in a cell by one of a class I and class II nuclear receptor. In a preferred embodiment, the nuclear receptor comprises a member of the nuclear receptor superfamily. In another preferred embodiment, the nuclear receptor is selected from the group consisting of ERα, ERβ, VDR, RARα, RARβ, and RARγ.

In yet another aspect of the present invention, there is provided a use of a corepressor polypeptide of the present invention to repress nuclear-receptor mediated transcription in a cell.

In yet another aspect of the present invention, there is provided a use of a corepressor polypeptide of the present invention to modulate steroid hormone signaling in a cell.

In yet another aspect of the present invention, there is provided a use of the corepressor polypeptide of the present invention to regulate gene expression in a cell.

In yet another aspect of the present invention, there is provided a use of a corepressor polypeptide of the present invention in an assay to select, for therapeutic purposes, compounds that modulate transcription of gene expression associated with the corepressor polypeptide.

In yet another aspect of the present invention, there is provided a method for assaying for compounds capable of modulating the activity of a corepressor polypeptide of the present invention or an active variant thereof to actively modify transcription of DNA. The method comprises (a) providing a corepressor polypeptide of the present invention or an active variant thereof; (b) contacting the corepressor polypeptide with a nuclear receptor in the presence and absence of the compound; and (c) measuring the modulation in activity of repression of DNA translation of the corepressor polypeptide.

In yet another aspect of the present invention, there is provided a method for assaying for compounds capable of affording selective recruitment of a corepressor polypeptide of the present invention in the presence of a ligand of a nuclear receptor, wherein the corepressor is operably interactable with the nuclear receptor to actively repress transcription of DNA in the presence of the ligand. In a preferred embodiment, the ligand comprises estrogen or an estrogen-like compound and the repressed DNA transcription products are implicated in hormone-dependent cancer.

Unless defined otherwise, the scientific and technological terms and nomenclature used herein have the same meaning as commonly understood by a person of skill in the art to which this invention pertains but should not be interpreted as limiting the scope of the present invention.

The term “LCoR corepressor” (ligand-dependent corepressor) as used herein is used to refer to novel corepressor polypeptides of the present invention. Use of the term LCoR, however, should not be interpreted as limiting the scope of the present invention to ligand-dependent corepressors only.

All publications, patents and patent applications are herein incorporated by reference in their entirety to the same extent as if each individual publication, patent or patent application was specifically and individually indicated to be incorporated by reference in its entirety.

### BRIEF DESCRIPTION OF THE DRAWINGS

FIGS. 1A-1D illustrate the LCoR corepressor gene (SEQ ID NO:1), transcript (SEQ ID NO:3) and protein structure (SEQ ID NO:2).

FIGS. 2A-2C illustrate that LCoR transcripts are widely expressed. FIG. 2A illustrates a plan of a Multiple Tissue expression Array (MTA) (Clontech) and the corresponding autoradiogram probed with an LCoR cDNA. FIG. 2B illustrates Northern blot of 15 μg of total RNA isolated from the cell lines indicated with LCoR or ubiquitin probes. FIG. 1C illustrates the in situ hybridization analysis of LCoR expression in human placenta.

FIGS. 3A-3C illustrate the interaction of LCoR and ER α in vivo. FIG. 3A illustrates Western analysis of LCoR in 20, 50 or 100 μg of extract from MCF-7, HEK293 and COS-7 cells using a rabbit polyclonal antipeptide antibody. FIG. 3B illustrates coimmunoprecipitation of LCoR with ERα. FIG. 3C illustrates bioluminescence resonance energy transfer (BRET) assays on COS-7 cells transiently cotransfected with plasmids expressing EYFP-ERα and rluc-LCoR or rluc-LCoR-LSKAA fusion proteins and treated with 10⁻⁷M β-estradiol (E2), hydroxytamoxifen (OHT), raloxifene, diethylstilbestrol (DES) or ethanol (−).

FIGS. 4A-4H illustrate LCoR interaction in vitro with ERα, ERβ, and VDR by GST pull-down assay.

FIGS. 5A-5K illustrate that LCoR is a nuclear receptor corepressor.

FIGS. 6A-6E illustrate that LCoR interacts directly with specific HDACs.

FIGS. 7A-7G illustrate that LCoR interacts with C-terminal binding proteins.

FIGS. 8 illustrate colocalization of LCoR and CtBP1 (A), CtBP2 (B), CtIP (C), Rb (D) and BMI1 (E) by confocal microscopy. Note that no fluorescence signal was seen in control experiments where specific antibody was removed or replaced with control IgG (data not shown). Magnifications 63×.

FIGS. 9 illustrate endogenous LCoR coimmunoprecipitates with CtBPs, CtIP, Rb and BMI1. Extracts of MCF-7 cells were immunoprecipitated with specific antibodies against CtBPs, CtIP, Rb, or BMI1. Precipitates were probed for immunoprecipitation of CtBP1, CtBP2, CtIP, Rb, or BMI1 as indicated, or coimmunoprecipitation of LCoR. Note that control immunoprecipitations were performed with goat or rabbit control IgGs in all cases. Controls are shown for CtBP and BMI1 only.

FIGS. 10 illustrate mutation of both CtBP binding sites of LCoR disrupts its interaction with CtBPs in MCF-7 cell extracts. MCF-7 cells were transfected with Flag-tagged wild-type LCoR or tagged LCoR mutated in one or both CtBP binding sites as indicated. Top panel: extracts and immunoprecipitations with anti-Flag antibody of transfected MCF-7 cells showing that tagged proteins are expressed at similar levels in all cases. Middle panel: control immunoprecipitation with anti-CtBP antibody and western blot showing that CtBP1 is expressed at similar levels in all cases. Bottom panel: coimmunoprecipitation of tagged LCoR derivative from extracts of transfected MCF-7 cells.

FIG. 11 illustrate subcellular localization and contribution of HDACs 3 and 6 to LCoR corepression A. Colocalization of endogenous HDAC6 and LCoR in MCF-7 nuclei by confocal microscopy (see Experimental Procedures for details). Note that no fluorescence signal was seen in control experiments where specific antibody was removed or replaced with control IgG. B. Colocalization of endogenous HDAC3 and LCoR in MCF-7 nuclei by confocal microscopy. Note that no fluorescence signal was seen in control experiments where specific antibody was removed or replaced with control IgG. C. Overexpressed HDAC6 is exclusively cytoplasmic in COS-7 cells. COS-7 cells were transfected with expression vectors for LCoR and HA-Flag-HDAC6, and expression patterns were visualized by confocal microscopy. Note that in contrast to 3A, LCoR was detected with Cy3-conjugated antibody and HA-Flag-HDAC6 with Cy2-conjugated antibody. A-C. Magnification 63×.

FIG. 12 illustrate coexpression of HDAC3 but not HDAC6 enhances LCoR corepression of ERα transactivation in COS-7 cells (E2; estradiol, 10 nM). A. Coexpression of HDAC6 enhances LCoR corepression in MCF-7 cells. B. Effect of HDAC inhibitor trichostatin A (TSA; 500 nM) on repression by LCoR and HDAC6 in MCF-7 cells. C. Effect of HDAC inhibitor trapoxin (TRAP; 50 nM) on repression by LCoR and HDAC6 in MCF-7 cells.

### DETAILED DESCRIPTION OF THE INVENTION

The invention provides a novel class of corepressor polypeptides having an amino acid sequence which comprises at least one LXXLL NR box motif, corepressor polypeptides having within their amino acid sequence at least two C-terminal binding protein interaction motifs, variants of the corepressor polypeptides, polynucleotides encoding for the corepressor polypeptides, expression vectors comprising the polynucleotides, host cells stably transformed with the expression vectors, antibodies that bind to the polypeptide corepressors, transgenic knock-out mice having disruption in an endogenous gene which encodes for the corepressor polypeptides, methods of modulating a cell, methods of inhibiting ligand-dependent transactivation in a cell, methods of repressing nuclear-receptor mediated transcription in a cell, methods of modulating steroid hormone signaling in a cell, methods of regulating gene expression, methods for assaying for compounds capable of modulating the activity of the corepressor polypeptides, and methods for assaying for compounds capable of affording selective recruitment of the corepressor polypeptides.

Therefore, in accordance with a first aspect of the present invention, there is provided a novel corepressor polypeptide, herein referred to as “LCoR”, which comprises at least one LXXLL nuclear receptor interacting NR box motif wherein L is leucine and X is any amino acid residue. Its function is distinct from those of NCoR and SMRT by virtue of the fact that it can be recruited to receptors through an NR box in the presence of an agonist. LCoR bears limited homology to other nuclear receptor coregulators. The LCoR corepressor thus represents a new class of nuclear receptor corepressor.

LCoR transcripts are widely expressed at variable levels in human adult and fetal tissues and in human cell lines. The highly homologous murine gene is expresses in 2-cell embryos, suggesting that LCoR functions from the earliest stages of embryonic development. LCoR is most highly expressed in the placenta, and at near term is predominately present in syncytiotrophoblasts. Receptors for estrogen, progesterone and glucocorticoids are expressed in the syncytiotrophoblast layer, which represents a barrier between the maternal and the fetal circulation and is a critical site of steroid hormone signaling, biosynthesis and catabolism (Pepe, G. J., and Albrecht, E. D. (1995) Endocrine Rev., 16, 608-48). The function of LCoR as an attenuator of nuclear receptor signaling indicating that it is an important modulator of steroid hormone signaling in syncytiotrophoblasts.

The sequence of LCoR contains a putative helix-loop-helix domain (HLH). It is noteworthy that multiple repeats of an HLH domain are required for high affinity site-specific DNA binding of Drosophila pipsqueak. Similarly, mutation of one of the two HLH motifs in the MBLK-1 gene strongly reduced site-specific DNA binding. The pipsqueak domain is homologous to motifs found once in a number of prokaryotic and eukaryotic proteins that interact with DNA, such as recombinases (Sigmund, T. and Lehmann; M. (2002) Dev. Genes Evol., 212, 152-57), suggesting that LCoR itself can interact with DNA.

Analysis of the interaction of LCoR with nuclear receptors by BRET, coimmunoprecipitation and GST pull-down assays indicates that LCoR can bind to receptor LBDs in a ligand-dependent or partially ligand-dependent manner. Moreover, the dependence of LCoR binding to ERα on the integrity of its LXXLL motif, and the integrity of ERα helix 12 indicates that LCoR associates with the same hydrophobic pocket in the LBD as p160 coactivators. However, while mutation of K362 (helix 3) disrupted binding of both LCoR and TIF-2.1, LCoR binding was more sensitive to mutation of amino acids at positions 347, 357 and 359 than TIF-2.1. LCoR binding was sensitive to the integrity of residue 347 of ERα, which lies outside binding groove residues 354-362 recognized by the NR box II peptide of TIF-2 (GRIP1; Shiau, A. K. et al. (1998) Cell, 95, 927-37), suggesting that LCoR recognizes an extended region of helix 3, and that LCoR residues outside the LXXLL motif contact the ERα LBD.

LCoR inhibited ligand-dependent transactivation by nuclear receptors in a dose-dependent manner up to 5-fold, and functioned as a repressor when coupled to the GAL4 DNA binding domain. While LCoR and p160 coactivators both bind in an agonist-dependent manner to coactivator binding pockets, several results indicate that the repression observed by LCoR was not simply a result of blockage of p160 recruitment. Rather, LCoR recruits multiple factors that act to repress transcription. While the HDAC inhibitor TSA abolished repression by LCoR of estrogen- and glucocorticoid-dependent transcription, the compound had little or no effect on repression of progesterone- or vitamin D-dependent transcription or repression by GAL-LCoR, indicating HDAC-dependent and -independent modes of action.

LCoR was observed to interact with HDACs 3 and 6, but not HDAC1 or HDAC4, in vitro, and interactions with HDACs 3 and 6 were confirmed in coimmunoprecipitations. Experiments indicate that HDACs 3 and 6 interact with distinct regions of LCoR in the C-terminal half of the protein. HDACs 3 and 6 are class I and II enzymes, respectively. Unlike other class II enzymes, HDAC6 contains two catalytic domains (Khochbin, S. et al. (2001) Curr. Opinion Genet Dev. 11, 162-6), and has not previously been associated with nuclear receptor corepressor complexes. Several biochemical studies to date have characterized different corepressor complexes associated with nuclear receptors, which include different HDACs (Rosenfeld M. G. and Glass, C. K. (2001) J. Biol. Chem., 276, 36865-68). Using SMRT affinity chromatography, HDAC3 was identified as a component of a multiprotein complex that also contained transducin β-like protein, TBL1, a homologue of the groucho corepressor. NCoR was also found to be part of a large complex purified by HDAC3 affinity chromatography (Wen et al, 2000). Studies to date suggest that NCoR and SMRT may interact with varying stability with distinct corepressor complexes that include multiple HDACs, indicating that compositions of individual corepressor complexes are not fixed.

LCoR was found to interact with the corepressor CtBP1 through tandem consensus CtBP-interaction motifs. Like LCoR, the sensitivity of repression by CtBPs to TSA is dependent on the promoter tested, indicative of HDAC-dependent and -independent modes of action (Chinnadurai, G. (2002) Mol. Cell, 9, 213-24). CtBP proteins interact with several different transcriptional repressors (Chinnadurai, G. (2002) Mol. Cell, 9, 213-24), including the nuclear receptor corepressor RIP140. The TSA-sensitive and -insensitive actions of LCOR are analogous to another CtBP-interacting repressor Ikaros, which is composed of distinct domains mediating repression by HDAC-dependent and -independent mechanisms. CtBP binding to Ikaros contributes to its HDAC-independent mode of action. CtBPs also associate with specific polycomb group (PcG) repressor complexes, and HDAC-independent repression of transcription by CtBP has been linked to its association with PcG complexes (Dahiya, A. et al. (2001) Mol. Cell, 8, 557-68). The present experiments indicate that LCoR also associates with components of PcG complexes. Therefore, in accordance with another aspect of the present invention there is provided an isolated corepressor polypeptide having within its amino acid sequence at least two C-terminal binding protein interaction motifs, said first C-terminal binding protein interaction motif comprising the sequence PLDLTVR, and said second C-terminal binding protein interaction motif comprising the sequence VLDLSTK, said polypeptide operably interactable with a C-terminal binding protein (CtBP) corepressor in a pathway to repress expression of DNA.

In accordance with another aspect of the present invention, there is provided an isolated polynucleotide coding for a novel corepressor polypeptide of the present invention or a variant thereof. There is also provided an expression vector comprising a polynucleotide encoding a corepressor polypeptide of the present invention or a variant thereof operably linked to a promoter for expression in a host cell. Preferred aspects of the expression vector and host cells stably transformed therewith are set out in the Examples and Materials and Methods as set out below.

The action of corepressors such as LCoR that recognize agonist-bound receptors indicates that there are signals that act to attenuate the consequences of hormone-induced receptor function. Such effects would provide a counterbalance to signals that augment hormone-induced transactivation; for example the stimulatory effects of MAP kinase signaling on ERα function (Kato, S. et al. (1995) Science, 270, 1491-4). Because LCoR acts to attenuate the function of agonist-bound receptors, posttranslational modification or LCoR and/or receptors will affect the relative affinities of LCoR and p160s for coactivator binding pockets. LCoR contains several putative phosphorylation motifs, including a number of MAP kinase sites in the region of the NR box, as well as potential sites for protein kinases A and C. Thus, LCoR's interaction with ligand-bound nuclear receptors can be modulated by phosphorylation. In addition, LCoR contains a consensus leptomycin B-sensitive nuclear export signal (LX₃LX₃LXIX₃L; a.a.149-164), indicating that its access to receptors is regulated by nuclear export under some conditions.

A rabbit polyclonal antipeptide antibody was raised against a portion of an LCoR sequence. Therefore, in accordance with another aspect of the present invention, there is provided an antibody that specifically binds to the corepressor polypeptide of the present invention. Preferred aspects of the antibodies of the present invention are set out in the Examples and Materials and Methods as set out below.

In accordance with another aspect of the present invention, there is provided a transgenic knock-out mouse comprising disruption in an endogenous gene which encodes for a corepressor polypeptide of the present invention, wherein a disruption has been introduced into its genome by a recombinant DNA construct stably integrated into the genome of said mouse or an ancestor thereof, wherein the disruption of the corepressor gene reduces expression of the corepressor polypeptide causing altered active transcription of DNA associated with the corepressor. Methods used to disrupt the gene and to insert the transgene into the genome of a mammalian cell, particularly a mammalian cell of a living animal are well known to those skilled in the art of trangsenic aminals. In the present invention, knock-outs can have a partial or complete loss of function in the endogenous gene.

In accordance with another aspect of the present invention, there is provided a method of modulating a cell comprising a gene which encodes for a corepressor polypeptide of the present invention comprising the steps of introducing into said cell the isolated polynucleotide having at least one variation in its sequence relative to that of the wild type, whereby expression of the corepressor polypeptide is modulated. Preferred aspects for varying the sequence are set out in the Examples and Materials and Methods as set out below.

In accordance with another aspect of the present invention, there are provided methods of inhibiting ligand-dependent transactivation in a cell by one of a class I and class II nuclear receptor, methods of repressing nuclear-receptor mediated transcription in a cell, methods of modulating steroid hormone signaling in a cell, methods of regulating gene expression in a cell, by use of the corepressor polypeptides of the present invention. Preferred aspects for the methods and uses are set out in the Examples and Materials and Methods as set out below.

In accordance with another aspect of the present invention, there is provided use of the polypeptide of the present invention in an assay to select, for therapeutic purposes, compounds that modulate transcription of gene expression associated with the corepressor polypeptide, as well as methods for assaying for compounds capable of modulating the activity of a corepressor polypeptide of the present invention or an active variant thereof to actively modify transcription of DNA. In a preferred aspect of the present invention, the method for assaying for compounds is used to identify compounds capable of affording selective recruitment of the corepressor polypeptide of the present in the presence of a ligand of a nuclear receptor, wherein the corepressor is operably interactable with the nuclear receptor to actively repress transcription of DNA in the presence of the ligand. In a preferred embodiment, the ligand comprises estrogen or an estrogen-like compound and the repressed DNA transcription products are implicated in hormone-dependent cancer. Preferred aspects for the methods and uses are set out in the Examples and Materials and Methods as set out below.

Materials and Methods

Isolation of LCoR cDNA Sequences

A yeast two-hybrid screen (2×10⁶ transformants; Clontech human fetal kidney cDNA Matchmaker library PT1020-1; Palo Alto, Calif. with an ERα-LBD bait in the presence of 10⁻⁶M estradiol yielded 10 His³⁰/LacZ⁺ colonies, of which 6 were dependent on estradiol for lacZ expression. 3 clones contained 1.2 kb inserts identical to coactivator AIB-1, and one contained an insert of 1.3 kb of LCoR sequence. 1.6×10⁶ human λgt11, prostate cDNA clones (Clontech, HL1131b) were screened for more LCoR sequence, yielding 5 clones containing LCoR sequences 1-1417, 462-1376, 704-1406, 1122-2915, 1214-3016. Multiple alignment of the different cDNA clones was performed (CAP program; INFOBIOGEN site http://www.infobiogen.fr). Homologies to ESTs and proteins were found using BLAST2 and PSI-BLAST, respectively, employing standard parameters and matrices.

Immunocytochemistry and In Situ Hybridization

MCF-7 cells were cultivated on collagen IV-treated microscope slides in 6-well plates, fixed with 2% paraformaldehyde for 15 min at room temperature, washed (3×) with PBS, and permeabilized with 0.2% Triton X100/5% BSA/10% horse serum in PBS. Cells were then incubated with α-LCoR (1:500), and αCtBP1 or αCtBP2 (1:50) in buffer B (0.2% Triton X100/5% BSA in PBS), for 1 h at room temperature. Cells were washed (3×) with PBS, and incubated with goat anti-rabbit-Cy2 and donkey anti-goat Cy3 (1:300) in buffer B for 1 h at room temperature. Slides were mounted with Immuno-Fluore Mounting Medium (ICN, Aurora, Ohio) and visualized using a Zeiss LSM 510 confocal microscope at 63× magnification. In situ hybridization was carried out using 443 bp sense and antisense LCoR probes, and a hybridization temperature of 60° C. and maximum wash conditions of 0.1×SSC at 65° C.

GST Pull-Down Assays and Immunoprecipitations

GST pull-down assays were performed as described (Eng, F.C.S. et al. (1998) J. Biol. Chem., 273, 28371-7), with the exception that assays performed with in vitro translated ER378 included two more washes made with the GST buffer containing 150 mM NaCl. For immunoprecipitations of tagged proteins, COS-7 cells in 100 mm dishes were transfected with 6 μg of HA-LCOR and/or 6 μg of HA-Flag-HDAC6 or with 6 μg of Flag-LCoR and/or 6 μg of HA-HDAC3 and pSG5 carrier. 48 h after transfection, cells were lysed 30 min at 40° C. in 1 ml of JLB (20 mM Tris-HCl, pH8, 150 mM KCl, 10% glycerol, 0.1% IGEPAL CA-630, and complete protease inhibitor cocktail; Boehringer-Mannheim, Laval, Qc). Cell debris were pelleted by centrifugation (14,000 rpm, 5 min), and proteins immunoprecipitated from 600 μl of supernatant by incubation for 1 h at 4° C. with 4 μg of α-Flag M2 antibody or polyclonal anti-HDAC3, followed by overnight incubation with protein A+G agarose or protein-A agarose beads for anti-Flag, and anti-HDAC3, respectively. Beads were washed (3×) with JLB. Bound immunocomplexes were boiled in Laemmli buffer, separated by 10% SDS/PAGE, and blotted on PVDF membrane with α-Flag M2-peroxidase, α-HDAC3, α-HA-peroxidase (1:500), and detected by enhanced chemiluminescence (NEN Life Science Products, Boston, Mass.). For immunoprecipitation of endogenous HDAC3 or HDAC6, MCF-7 cells in 150 mm dishes were lysed in 2 ml of JLB. Supernatants were cleared, incubated with 4 μg of αHDAC6 or αHDAC3 or control rabbit IgG in the presence of protein A agarose, and Western blotted as above. For ERα or CtBP, MCF-7 cells were lysed in 2 ml of 150 mM NaCl/10 mM TRIS-HCl pH 7.4/0.2 mM Na orthovanadate/1 mM EDTA/1 mM EGTA/1% Triton-100X/0.5% IGEPAL CA-630/protease inhibitor cocktail, and immunoprecipitated as above with 4 μg of αCtBP or αERα antibodies, or corresponding control IgG in the presence of protein A or protein A+G agarose, respectively. Dilutions of specific antibodies used for Western blotting were: LCoR, HDAC3, and HDAC6 (1:1000), CtBP1, CtBP2 and ERα(1 :100).

BRET Assays

COS-7 cells in 6-well plates were transfected with 250 ng of LCOR-rluc alone or with 2.5 μg of ERα-EYFP, and treated 24 h later with 10⁻⁷M estradiol, or OHT for 18 h. Cells were washed (2×) with PBS and harvested with 500 μl of PBS-5 mM EDTA. 20,000 cells (90 μl) were incubated with 5 μM final of coelenterazine H in 96-well microplates (3610, Costar, Blainville, Qc). Luminescence and fluorescence signals were quantified with a 1420 VICTOR²-multilabel counter (Wallac-Perkin Elmer, Boston, Mass.), allowing sequential integration of signals detected at 470 nm and at 595 nm. Readings were started immediately after coelenterazine H addition, and 10 repeated measures were taken. The BRET ratio was defined as [(emission at 595)−(emission at 470)×Cf]/(emission 470), where Cf corresponded to (emission at 470/emission at 595) for the rluc-LCoR expressed alone in the same experiments.

Antibodies

A rabbit polyclonal antipeptide antibody was raised against LCoR a.a 20-36 (QDPSQPNSTKNQSLPKA; SEQ ID NO:4) fused to keyhole limpet hemocyanin, and purified over a peptide affinity column (Bethyl Laboratories, Montgomery Tex.). Mouse monoclonal α-ERα (sc-8005), rabbit polyclonal α-CtBP (sc-11390), goat polyclonal α-CtBP1 (sc-5963), goat polyclonal α-CtBP2 (sc-5967), protein A-agarose and protein A+G-agarose were from Santa Cruz Biotechnology (Santa Cruz, Calif., USA). Rabbit polyclonal α-HDAC3 (382154) was from Calbiochem (San Diego, Calif., USA). Rabbit polyclonal α-HDAC6 was raised against the C-terminal third of HDAC6. Cy3-donkey polyclonal α-goat (705-165-147) and Cy2-goat polyclonal α-rabbit (711-225-152) were purchased from Jackson ImmunoResearch (West Grove, Pa., USA). Mouse monoclonal α-Flag M2 (F3165), and α-FLAG M2 HRP-conjugate (A-8592), monoclonal α-rabbit HRP conjugate (A2074), rabbit polyclonal α-goat HRP conjugate (A5420) and goat polyclonal α-mouse HRP conjugate (A9917) were from Sigma (St. Louis, Mo.). Mouse monoclonal antibody α-HA HRP conjugate was purchased from Roche Diagnostics (Laval, Qc)

Recombinant Plasmids

GST fusions in pGEX2T of ERα-LBD, TIF-2.1, and hVDR-LBD, HG1, hPR, ERE3-TATA/pXP2, 17 mer5-tk/pXP2, GAL4-DBD(1-147)/pSG5, TIF-2.1/pSG5, TIF2/pSG5 have been described (Aumais, J. et al. (1996) J. Biol. Chem., 271, 12568-12577; Lee, H.S. et al. (1996) J. Biol. Chem. 271, 25727-25730; Eng, F.C.S. et al. (1998) J. Biol. Chem., 273, 28371-7). ERα-mAF2 was constructed by point mutagenesis of L539 and L540 to A residues. ERα-EYFP was constructed by insertion of an ERα cDNA lacking a stop codon into EcoRI and BamHI sites of pEYFP-CMV. For ER378/pSG5, a.a 1-378 of ERα was amplified using 5′ primer 5′CCGGMTTCCGGATGACCATGACCCTCCAC3′ (SEQ ID NO:5) and 3′ primer 5′CGGGATCCCGTCAAAGGTGGACCTGATCATG3′ (SEQ ID NO:6) and subcloned in EcoRI/BamHI digested pSG5. The GRE5 promoter was excised with XbaI and BamHI and subcloned to the SmaI/BgIII sites of pXP2 to make GRE5/pXP2, and VDRE3tkCAT was digested with BamHI and BgIII and VDRE3tk subcloned into pXP2 to give VDRE3tk/pXP2. ERα mutants T347A, N359S, and H356R were identified by sequencing of clones of the LBD mutagenized by PCR amplification. Mutagenized LBD sequences were subcloned as Hindlll-Xbal fragments into Hindlll-Xbal digested pGEX2T-ERα-LBD. The 475-918bp region of LCoR was amplified with 5′ primer 5′CCGGAATTCCGGCCCGGGCATGAGACAGTCCCTG-GGTCTC3′ (SEQ ID NO:7) and a 3′ primer with an endogenous KpnI site (position 918 bp) 5′TTCTTGGAGGTACCCCATCA3′ (SEQ ID NO:8) and inserted into 918-2915 LCoR/pSG5 digested with EcoRI and KpnI to create 475-2915 LCoR, which contains a full-length ORF (subsequently called LCoR/pSG5), and into pGEM-T-easy (Promega, Madison, Wis.) to create probes for in situ hybridization. The PCR fragment was verified by sequencing. LCoR/pSG5 was digested with SfrI and BamH1 and subcloned in BamHI site of GAL4DBD/pSG5 to create GAL4-LCoR/pSG5. Point mutagenesis of LSKLL to LSKAA at position 53, and deletion of PLDLTVR (a.a. 64-70; m1) and VLDLSTK (a.a. 82-88; m2) were made by QuickChange Site-Directed Mutagenesis Kit (Stratagene, La Jolla, Calif.). For GST-LCoR and GST-LSKAA, PCR amplification of LCoR or LCOR-LSKAA was performed with 5′CGCGGATCCGCGATGCAGCGMTGATCCM3′ (SEQ ID NO:9), and 5′GGMTTCCCTACTCGTTTTTTGATTCATT3′ (SEQ ID NO:10), digested with BamHI and EcoRI, and inserted into pGEX2TK. For LCoR-rluc, LCoR or LCOR-LSKAA were amplified with 5′ primer 5′CTAGCTAGCCACCATGCAGCGMTGATCCM3′ (SEQ ID NO:11) and 3′ primer 5′CTAGCTAGCCGCTCGTTTTTTGATTCATT3′ (SEQ ID NO:12). PCR products were digested with Nhe1 and cloned into pRL-CMV (Promega), and verified by sequencing. For HA-LCoR, HA-LSKAA, Flag-LCoR and Flag-LSKAA, cDNA sequences from LCoR/pSG5 or LSKAA/pSG5 were amplified using 5′CGGMTTCCAGCGMTGATCCMCM3′ (SEQ ID NO:13) and 5′CGCGGATCCGCGCTACTCGTTTTTTGATTCATT3′ (SEQ ID NO:14), digested with EcoRI and BamHI and inserted into the corresponding sites of HA/pCDNA3 or Flag/pCDNA3.

Cell Culture and Transfections

All cell lines were cultured under the recommended conditions. COS-7 cells grown in 6-mm plates in DMEM without phenol red, supplemented with 10% FBS were transfected in medium without serum with lipofectamine 2000 (Invitrogen, Burlington, Ont.) with 100 ng of nuclear receptor expression vectors as indicated, 200 ng of TIF-2 or TIF-2.1, as indicated, 250 ng of reporter plasmid, 250 ng of infemal control vector pCMV-βgal, and various concentrations of LCoR/pSG5 or LCoR-LSKAA/pSG5 expression vectors and pSG5 carrier. Medium was replaced 24 h after transfection by a medium containing charcoal-stripped serum and ligand (100 nM) and TSA (3 μM) for 18 h, as indicated. Cells were harvested in 200 λl of reporter lysis buffer (Promega).

Northern Blotting

A human Multiple Tissue Expression array (MTE array; Clontech; 7775-1) was probed with a 1.3 kb LCOR cDNA fragment by prehybridization in ExpressHyb buffer (Clontech) at 65° C. for 30 min and hybridization in the same solution containing 10⁷ cpm of the ³²P-labeled LCoR probe at 65° C. overnight, washed according to the manufacture's protocol. An ubiquitin probe was used as a positive control; 15 μg of total RNA was extracted cells with TRIZOL (Invitrogen, Burlington, Ont.) and electrophoresed on a 1% agarose gel containing 6.3% formaldehyde, 20 mM MOPS (pH 7.0), 15 mM sodium acetate, and 1 mM EDTA. RNAs were blotted on Hybond−N+ (Amersham, Baie d'Urfe, Quebec) and hybridized as for the MTE array.

The present invention will be more readily understood by referring to the following examples which are given to illustrate the invention rather than to limit its scope.

### EXAMPLE 1

### Identification of LCoR

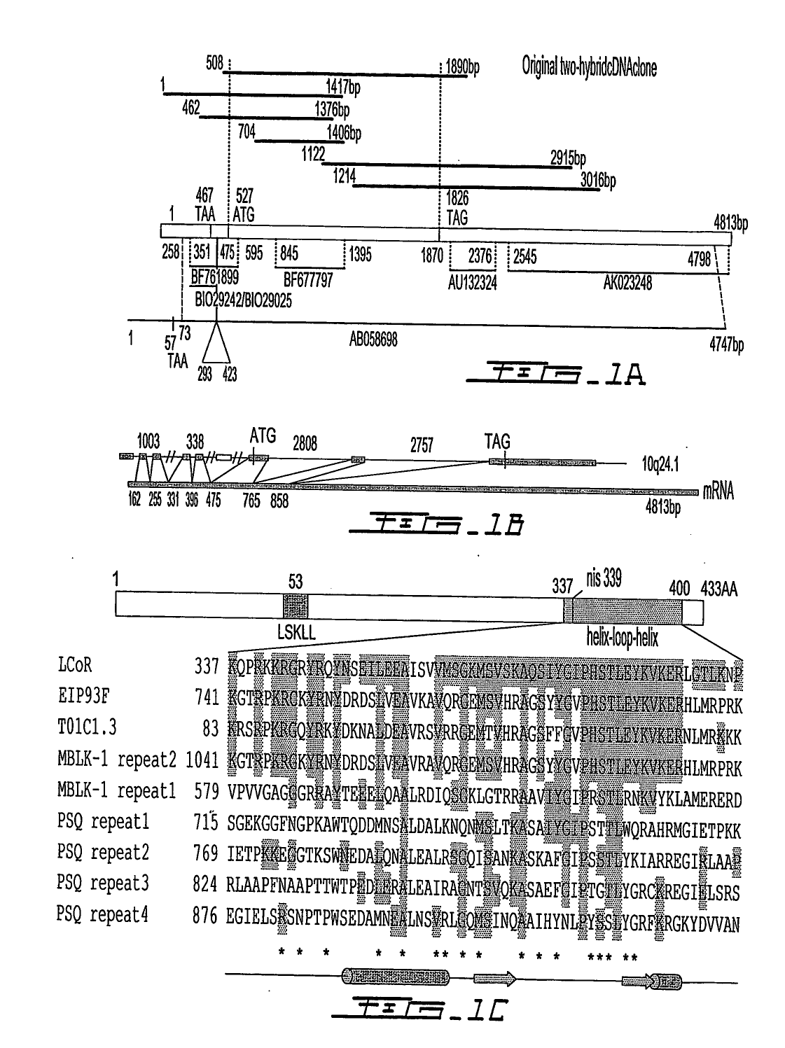

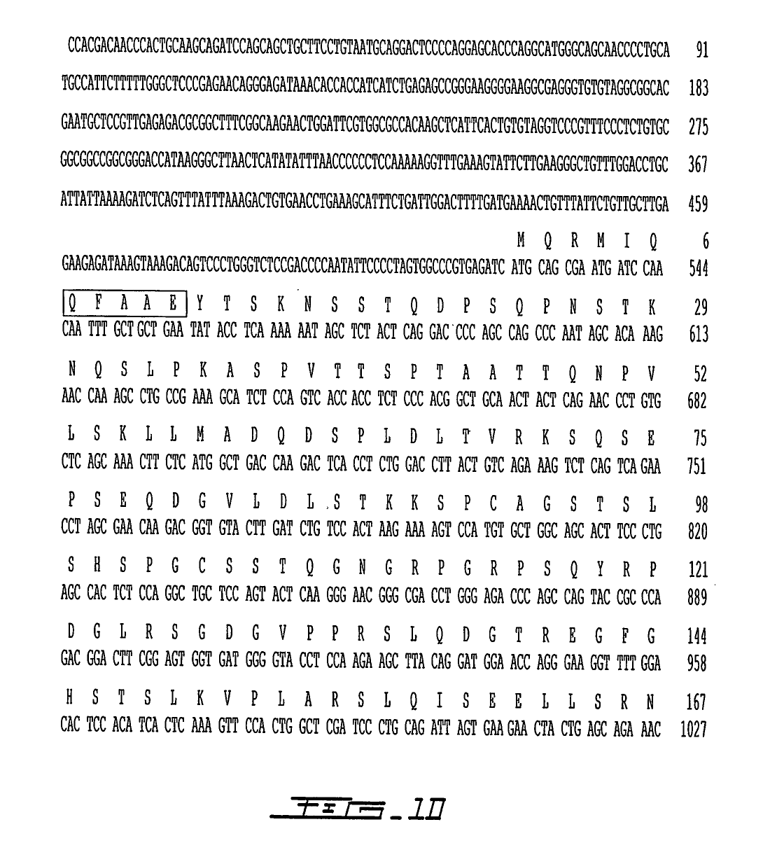

LCoR of FIG. 1 was isolated from a yeast two-hybrid library as a cDNA containing a 1299 nucleotide open reading frame (433 amino acid; 47,006 Da; FIGS. 1A and D) encoding a protein that interacted with the ERα LBD in an estradiol-dependent manner. Additional cDNAs were obtained from a human prostate cDNA library, and several expressed sequence tags (ESTs; FIG. 1A). In FIG. 1A, the LCoR two-hybrid cDNA clone (top), and clones isolated from a prostate cDNA library (below) are shown. LCoR ESTs are shown below the composite 4813 bp cDNA sequence (white bar). The open-reading frame of LCoR is indicated by the start codon and the downstream stop codon. The first upstream in-frame stop codons are also indicated. Human ESTs were identified using the INFOBIOGEN site (http://www.infobiogen.fr/services/analyseq/cgi-bin/blast2₁₃ in.pl). ESTs BF761899, BF677797, AU132324, AK023248, and B1029242/B1029025 are from adult colon, adult prostate, NT2 teratocarcinoma cell line, and adult marrow cDNA libraries, respectively. A 4747 bp cDNA (AB058698) identified from a human brain library, containing an extra 5′UTR exon is indicated at the bottom. Human sequences were also highly homologous (˜95%) to several mouse ESTs, including multiple clones from a two-cell embryo, indicating that LCoR is expressed from the earliest stages of mammalian development.

The 4.8 kb of cDNA sequence encompasses seven exons on chromosome 10q24.1, including 4 short 5′UTR exons that contain several in-frame stop codons (FIG. 1B). FIG. 1B illustrates the structure of the LCoR gene deduced using the Draft Human Genome Browser (http://genome.ucsc.edu/goldenPath.html). The extra 5′UTR exons present in the human brain cDNA AB058698 are indicated as white bars. Intron sizes are indicated where known. A human brain EST contains a single exon insert that lengthens the 5′UTR without extending the open reading frame, and contains an upstream stop codon (FIGS. 1A and B). The initiator ATG of LCoR lies within a consensus Kozak sequence RNNatgY.

LCoR of FIG. 1 bears only limited resemblance to known coregulators. There is a single LXXLL motif (NR box) at amino acid 53, and a PRKKRGR motif at amino acid 339 that is homologous to a simple nuclear localization signal (NLS) of the SV40 large T antigen-type. The NLS lies at the N-terminus of a putative helix-loop-helix domain (FIGS. 1C and D, SEQ ID NO:1-3), which is 48, 48, and 43% homologous to motifs encoded by the Eip93F, T01C1.3, and MBLK-1 genes of Drosophila, C. elegans, and Honeybee (Apis mellifera), respectively (FIG. 1C; SEQ ID NO:3). The domain also bears 35% homology to the pipsqueak motif (PSQ) repeated four times in the DNA binding domain of the Drosophilatranscription factor pipsqueak. FIG. 1C is a schematic representation of an LCoR corepressor protein of the present invention. The NR box LSKLL, nuclear localization signal (NLS), and putative helix-loop-helix (HLH) domain are indicated. The homologies of the HLH with other proteins are shown, with asterisks indicating positions of amino acid similarity. Existence of the HLH was predicted using Psired (http://bioinf.cs.ucl.ac.uk) and Network Protein Sequence @nalysis (http://pbil.ibcp.fr).

In FIG. 1D, the sequence of 1826 bp of a LCoR cDNA (SEQ ID NO:1) and complete predicted 433 amino acid protein (SEQ ID NO:2) sequences are presented. The LSKLL is boxed, the NLS is underlined, and the helix-loop-helix domain is highlighted.

### EXAMPLE 2

### LCoR is Widely Expressed in Fetal and Adult Tissues

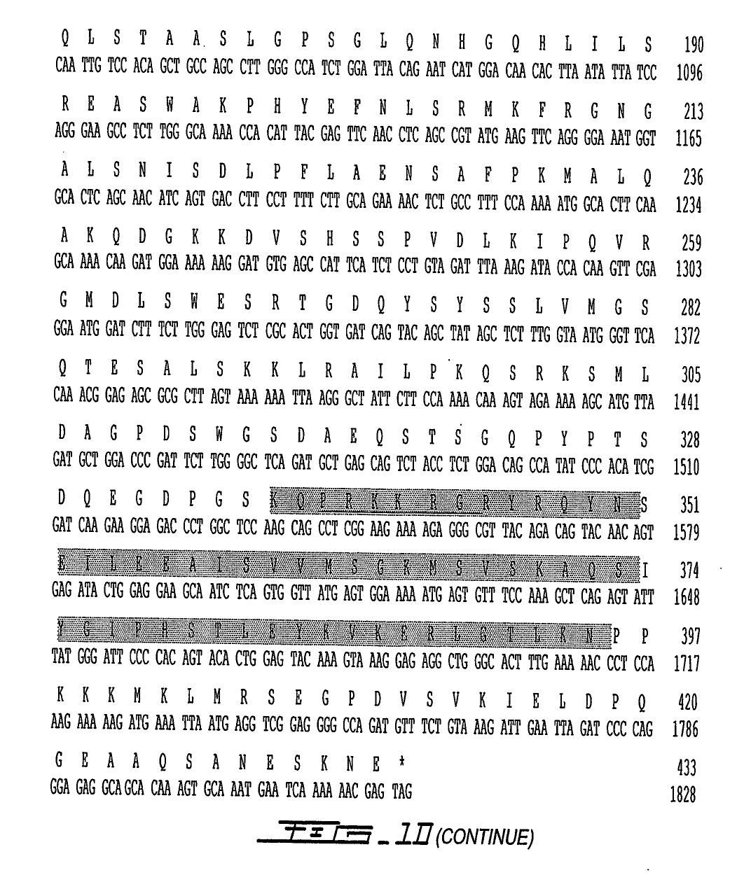

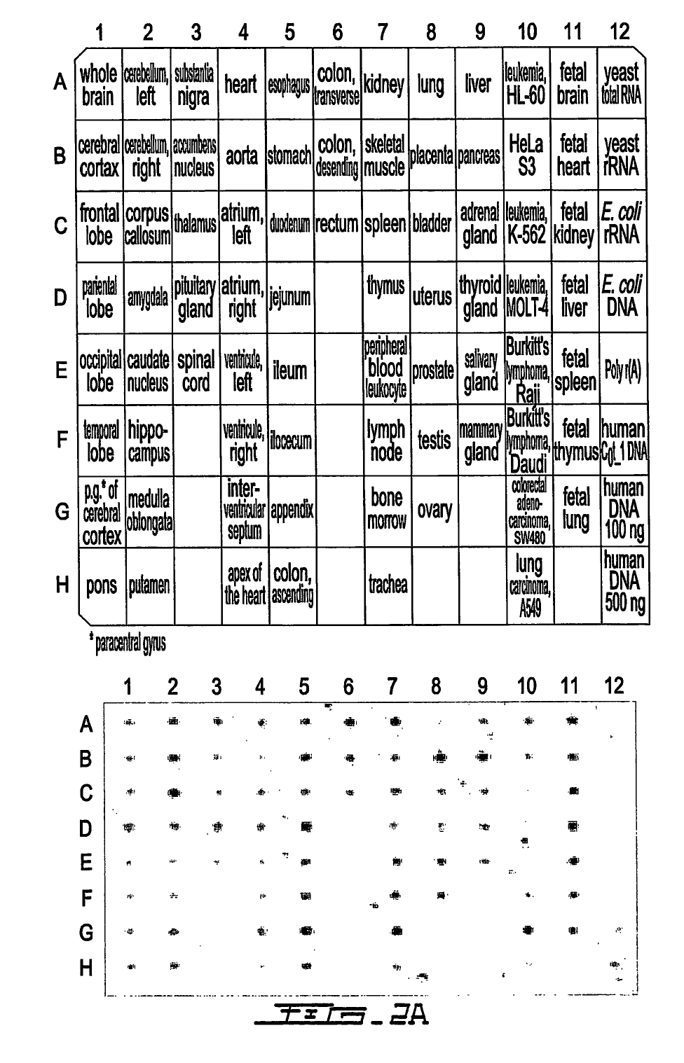

LCoR transcripts are widely expressed at varying levels in human adult and fetal tissues (FIGS. 2A-2C). Highest expression is observed in placenta, the cerebellum and corpus callosum of the brain, adult kidney and a number of fetal tissues. FIG. 2A illustrates a Multiple Tissue expression Array (MTA) (Clontech) and the corresponding autoradiogram probed with an LCoR cDNA. Probing the array with an ubiquitin probe as a positive control gave the results predicted by the manufacturer.

LCoR transcripts were also detected in a wide variety of human cell lines (FIG. 2B), with highest levels of expression observed in intestinal Caco-2 cells, and embryonic HEK293 kidney cells. FIG. 2B illustrates a Northern blot of 15 μg of total RNA isolated from the cell lines indicated with LCoR or ubiquitin probes. SCC4, SCC9, SCC15, and SCC25 are human head and neck squamous carcinoma lines; MDA-MB231, MDA-MB361, and MCF-7 are human breast carcinoma cell lines; HeLa, LNCaP, and CaCo-2 are human cervical, prostate, and colon carcinoma lines, respectively. HEK293 cells are derived from human embryonic kidney and COS-7 from monkey kidney. While LCoR transcripts were abundant in MDA-MB361 breast carcinoma cells, expression was weaker in MDA-24-MB231 and MCF-7 breast cancer lines (FIG. 2B). Along with the EST data cited above, these results indicate that LCoR transcripts are widely expressed throughout fetal development and in the adult.

Given the robust expression of LCoR transcripts in placenta, and the complex placental steroid physiology, LCoR expression was investigated further by in situ hybridization analysis of a section of human placenta (FIG. 2C). FIGS. 2C(i) and 2C(ii) are bright and dark field photomicrographs of the chorionic villi (CV) of a near term placenta (36 weeks) probed with a 443 b ³⁵S-labeled LCoR antisense probe (Magnification 20×). The inset of FIG. 2C(ii) illustrate dark field photomicrograph of a section probed with a control LCoR sense probe. FIGS. 2C(iii) and 2C(iv) are as in (i) and (ii) except at 40× magnification (Syn, syncytiotrophoblast; cm, chorionic mesoderm). The results reveal that LCoR is predominantly expressed in the syncytiotrophoblast layer of terminally differentiated cells, which acts as a barrier between maternal circulation and the fetus whose function is critical for controlling maternal hormonal signals that modulate fetal metabolism and development (Pepe, G. J., and Albrecht, E. D. (1995) Endocrine Rev., 16, 60848).

### EXAMPLE 3

### Agonist-Dependent Interaction of LCoR and ERα In Vivo

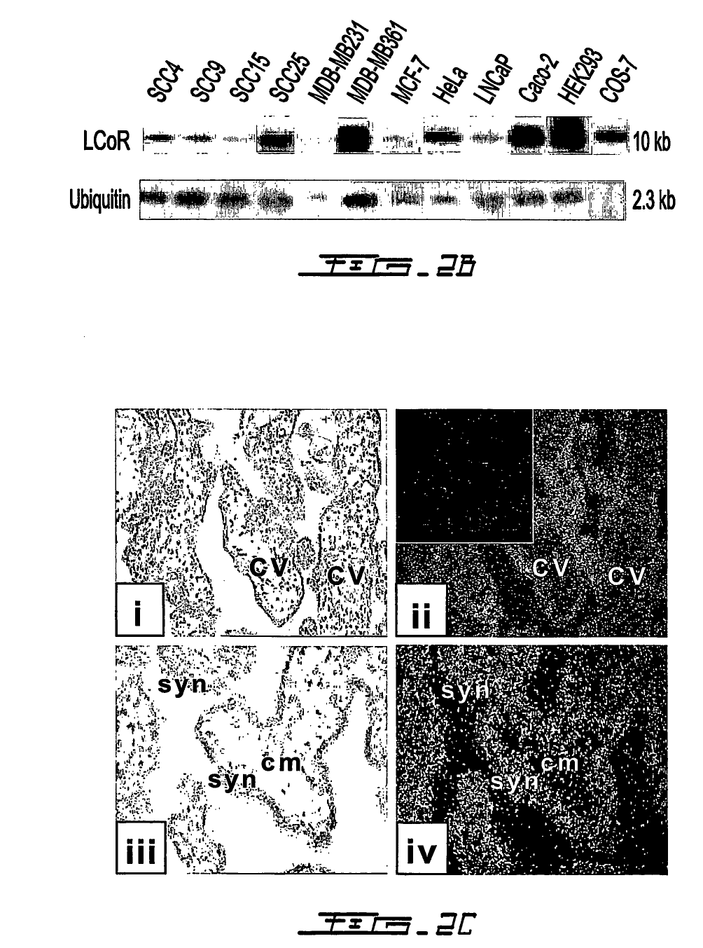

An affinity-purified antibody developed against an LCoR peptide detected a protein of approximately 50 kDa in MCF-7, HEK293, and COS-7 cell extracts (FIG. 3A), in excellent agreement with cDNA cloning data. FIG. 3A illustrates a Western analysis of LCoR in 20, 50 or 100 μg of extract from MCF-7, HEK293 and COS-7 cells using a rabbit polyclonal antipeptide antibody. The antibody also specifically detected several LCoR fusion proteins and deletion mutants. Immunocytochemical studies with the antibody in all three lines revealed a nuclear protein (see below). Consistent with two-hybrid cloning, endogenous LCoR coimmunoprecipitated with endogenous ERα in an estradiol-dependent manner from MCF-7 cell extracts (FIG. 3B). Western blots (WB) of ERα (left) and LCoR (right) in immunoprecipitates of ERα with control mouse IgG or mouse monoclonal anti-ERα antibody from extracts of MCF-7 cells treated for 4 h with vehicle (−) or estradiol (E2) as illustrated in FIG. 3B. No immunoprecipitation of ERα or LCoR was observed when anti-ERα antibody was replaced by control IgG (FIG. 3B). Reduced ERα expression after estradiol treatment is consistent with enhanced turnover of the receptor observed in hormone-treated MCF-7 cells.

Interaction of ERα and LCoR in vivo was further tested by bioluminescence resonance energy transfer (BRET) in living COS-7 cells transiently cotransfected with plasmids expressing ERα-EYFP and LCoR-rluc fusion proteins. Consistent with coimmunoprecipitations, treatment with estradiol or diethylstilbestrol (DES) enhanced BRET ratios 2.5 to 3-fold (FIG. 3C), consistent with agonist-dependent interaction of LCoR and ERα, whereas treatment with antiestrogens 4-hydroxytamoxifen (OHT) or raloxifene had no significant effect. FIG. 3C illustrates Bioluminescence resonance energy transfer (BRET) assays on COS-7 cells transiently cotransfected with plasmids expressing EYFP-ERα and rluc-LCoR or rluc-LCoR-LSKAA fusion proteins and treated with 10⁻⁷M β-estradiol (E2), hydroxytamoxifen (OHT), raloxifene, diethylstilbestrol (DES) or ethanol (−). BRET ratios were calculated as described in experimental procedures. The data shown represent the mean±SEM of 3 experiments. Moreover, mutation of the NR box of LCoR to LSKAA largely disrupted hormone-dependent interaction and reduced hormone-independent interaction of the two proteins by approximately two-fold (FIG. 3C), indicating that the LCoR LXXLL motif is essential for ligand-dependent interaction with ERα.

### EXAMPLE 4

### Interaction of LCoR with Nuclear Receptor Ligand-Binding Domains In Vitro

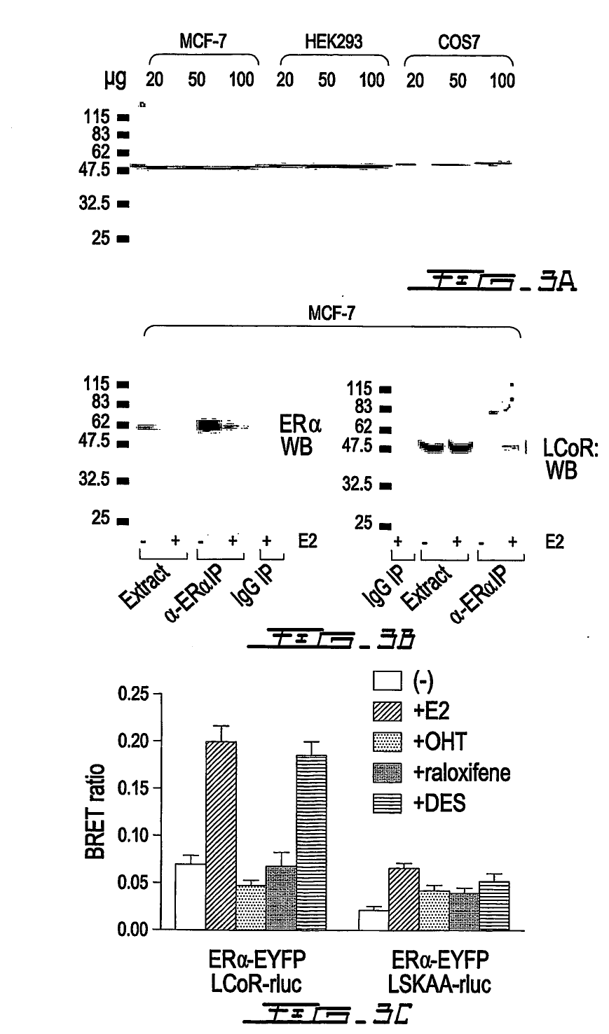

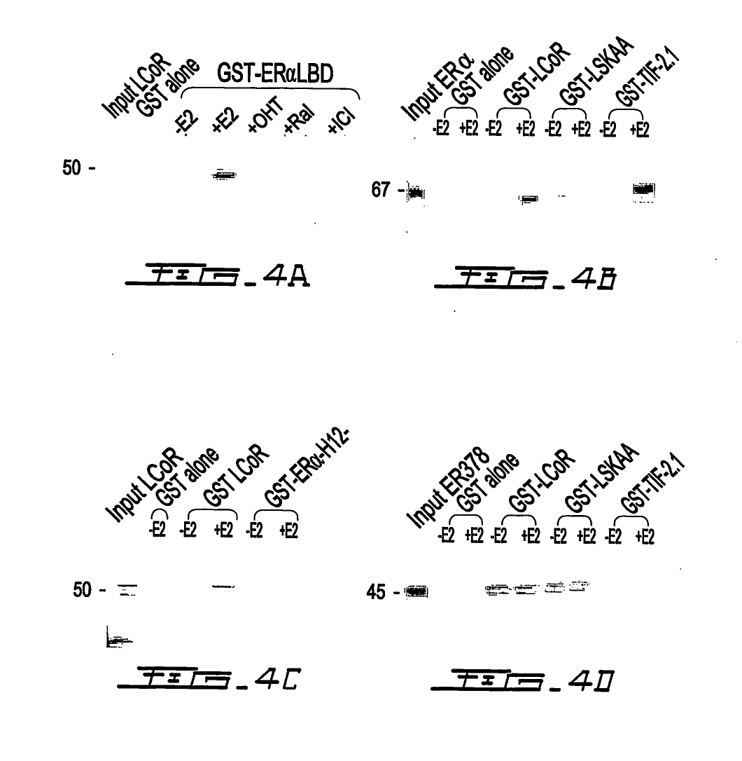

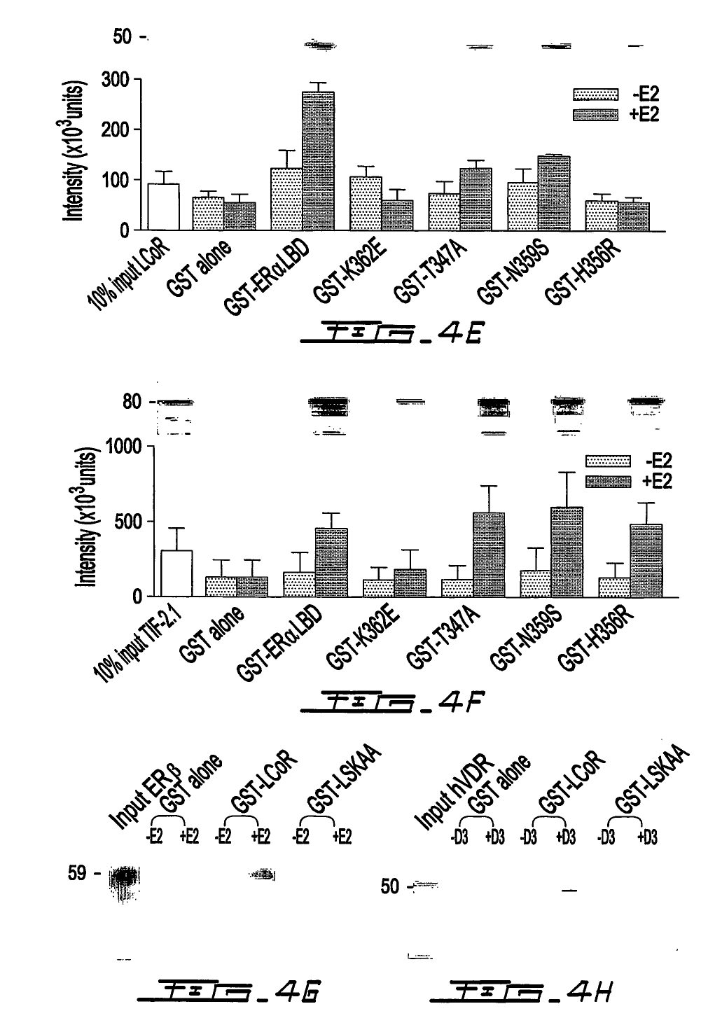

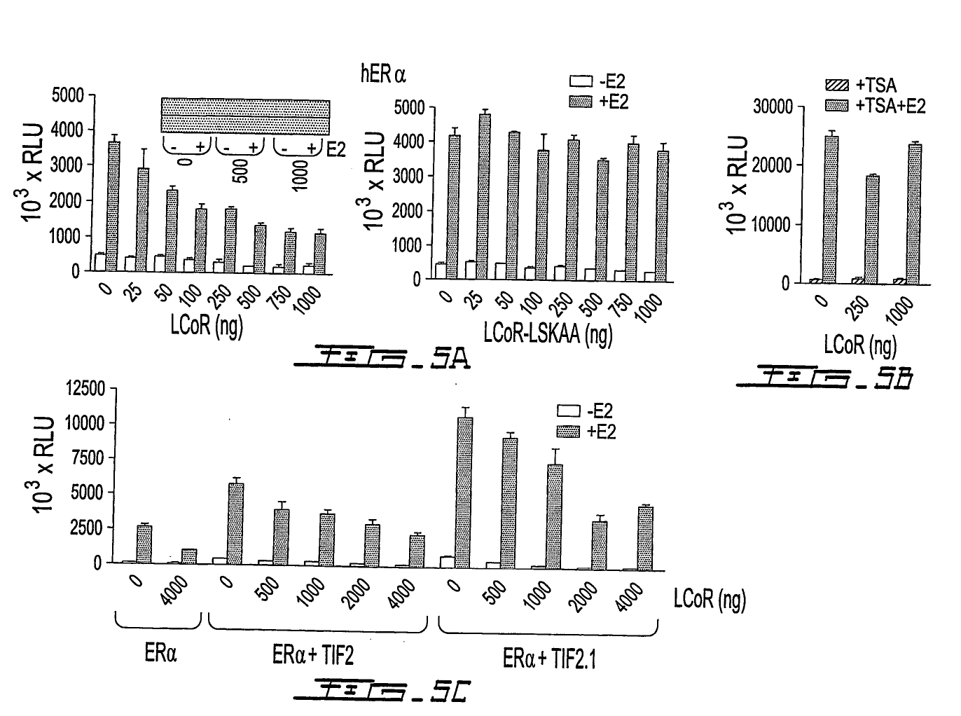

In vitro translated LCoR selectively bound to the ERα LBD fused to GST (GST-ERα-LBD) in a partially estrogen-dependent manner (FIG. 4A). In FIG. 4, Estradiol (E2), hydroxytamoxifen (OHT), raloxifene (Ral), and ICI164,384 (ICI), vitamin D3 (D3) were added to 10⁻⁶ M as indicated. Inputs (lanes 1) represent 10% of the amount of labeled protein used in assays. FIG. 4A illustrates ligand-dependent interaction of in vitro-translated LCoR with GST-ERα LBD. FIGS. 4B and 4D illustrate the interaction of in vitro translated ERα (HEG0; B) or ER378 (D) with GST fused to LCoR, LCoR-LSKAA or TIF2.1 as indicated. FIG. 4C illustrates the interaction of LCoR with GST-ERα or a helix 12 mutant (ERα-mAF-2). FIGS. 4E and 4F illustrate the interaction of GST fusions of wild-type ERα LBD or LBD mutants T347A, H356R, N359S, and K362A with LCoR (E) or TIF-2.1 (F). Histograms of results of triplicate experiments are shown. Bands were quantitated using the FluorChem digital imaging system and AlphaEaseFC software (Alpha Innotech Corp, San Leandro, Calif.). FIGS. 4G and 4H illustrate the Interaction of ERβ (G) and VDR (H) with GST-LCoR and GST-LSKAA.

Consistent with BRET analyses, antiestrogens OHT, raloxifene, or ICI 164,384 did not induce interaction of LCoR with ERα (FIG. 4A), and hormone-dependent binding of ERα was abolished by mutation of the LCoR NR box (LSKAA; FIG. 4B). Similar results were obtained with GST-ERα fusions and in vitro translated LCoR-LSKAA. Furthermore, double point mutation of the ERα AF-2 domain in helix 12 (L539A, L540A; mAF-2) abolished ligand-dependent binding of LCoR (FIG. 4C). ERα was truncated to amino acid 378 (ER378), leaving regions A-D and the N-terminal third of the LBD (FIG. 4D), or to amino acid 282 in region D (HE15) or 180, which encodes the A/B domain. While ER378 bound specifically to GST-LCoR, but not TIF-2.1, in a hormone-independent manner (FIG. 4D), no such interaction was observed with HE15 or the A/B domain, suggesting that residues contributing to ligand-independent interaction with LCoR are located between ERα amino acids 283 and 377.

Interaction of LCoR with helix 3 was further probed using GST fusions of ERα point mutants T347A, H356R, N359S, and K362E. Helix 3 forms a critical part of the static region of the coactivator binding pocket (Shiau, A. K. et al. (1998) Cell, 95, 927-37), and the integrity of lysine 362 at the C-terminus of helix 3 (Brzozowski, A. M. et al. (1997) Nature, 389, 753-8) is essential for ligand-dependent binding of p160 coactivators. While the K362A mutation disrupted both TIF-2.1 and LCoR binding, mutations T347A, H356R, N359S had minimal effect on interaction of TIF-2.1, but partially or completely abolished binding of LCoR (FIGS. 4E and F). The above data indicate that LCoR and TIF-2.1 recognize overlapping binding sites, although LCoR interacts with residues on helix 3 that are distinct from those recognized by TIF-2.1.

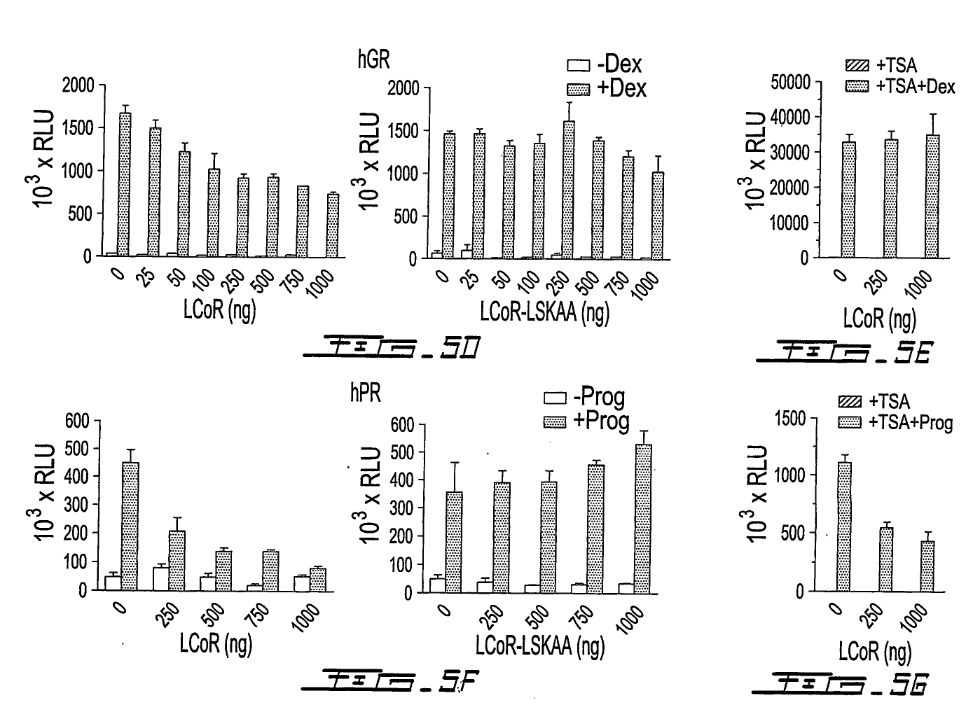

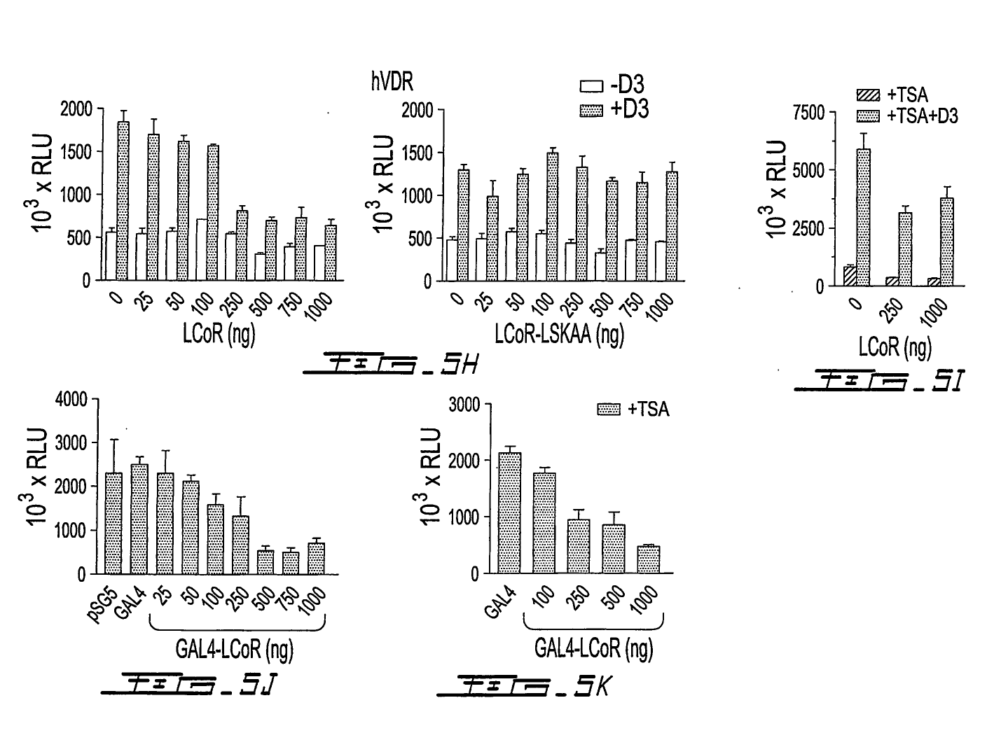

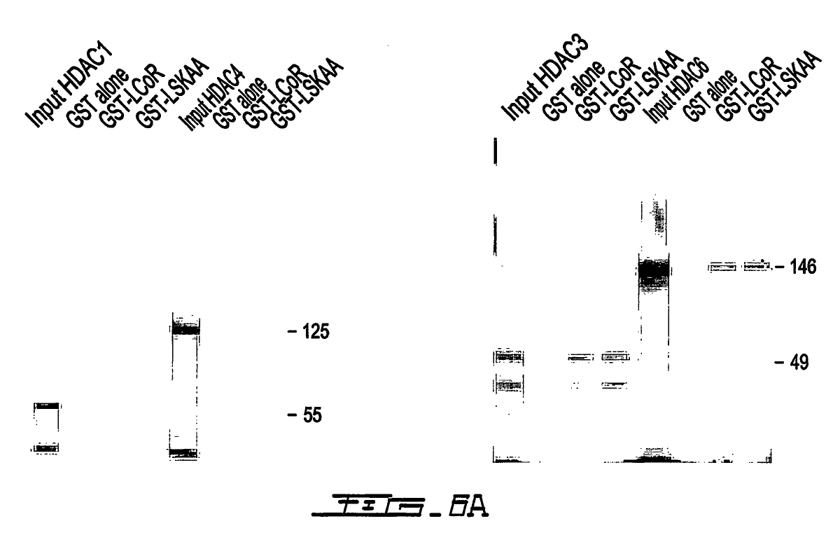

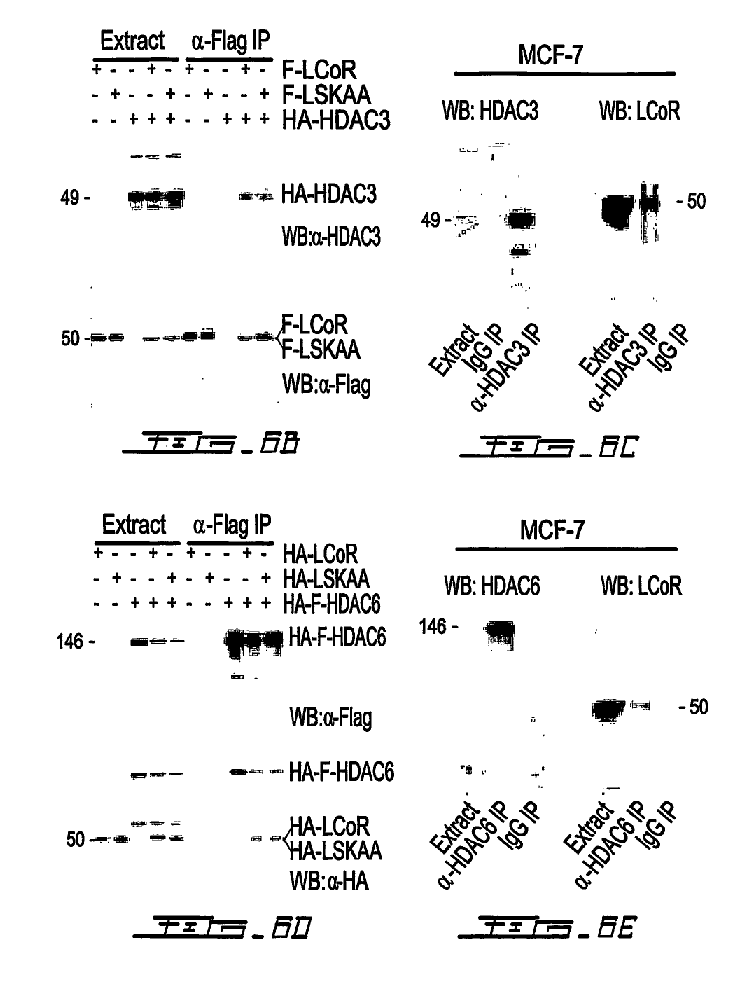

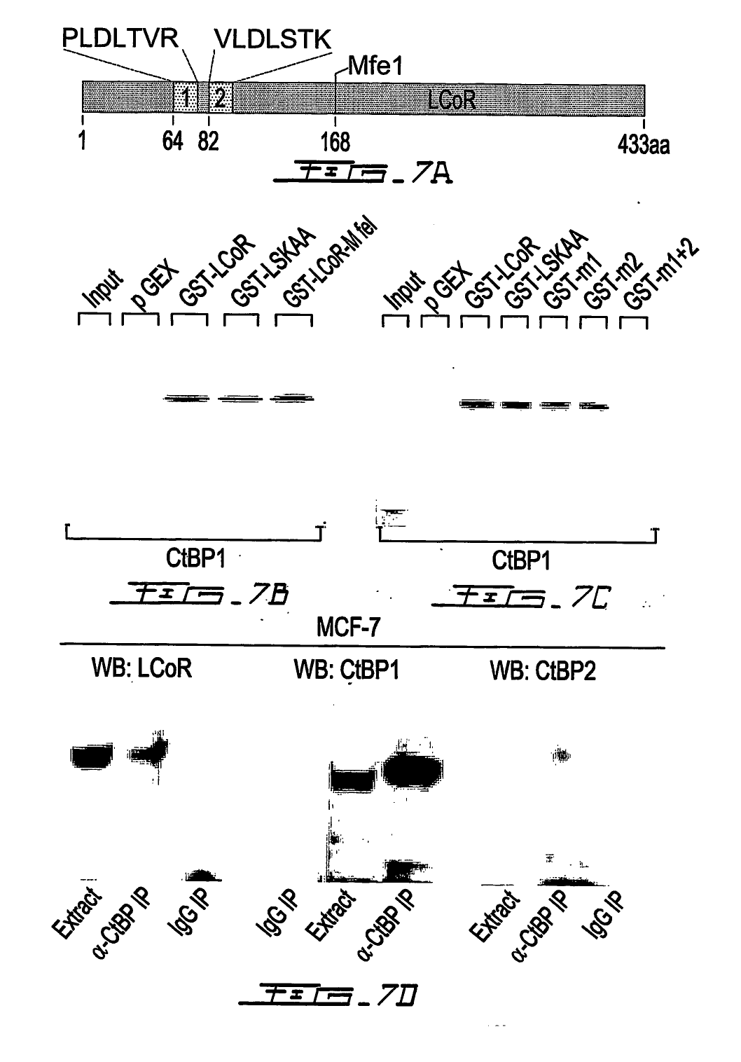

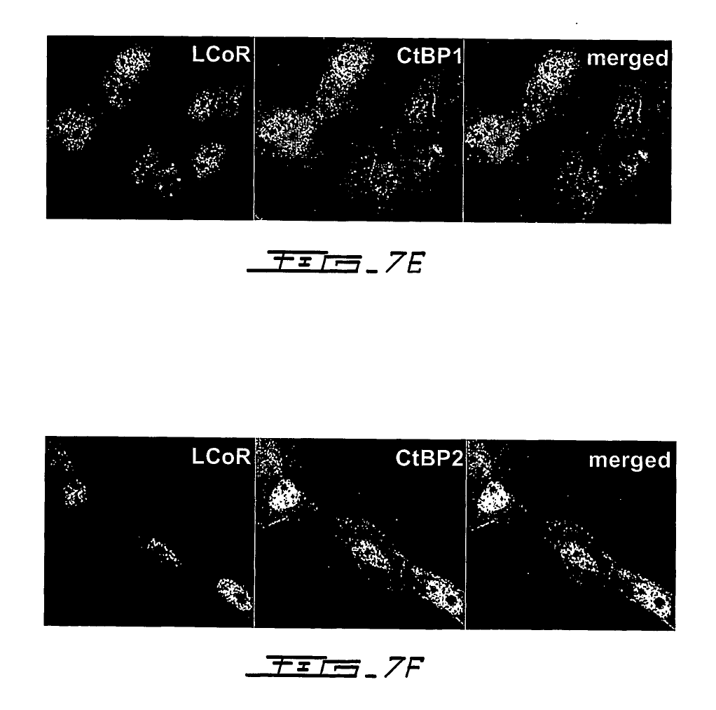

Binding of LCoR to other nuclear receptors was also analyzed by GST pull-down assays, which showed that LCoR also bound LBDs of ERβ, VDR, RARs α, β, and γ, and RXRα in a ligand-dependent manner (FIG. 5G and H). Taken together, the above results indicate that LCoR can bind to the LBDs of several nuclear receptors in a hormone-dependent or partially hormone-dependent manner, and the interaction of LCoR with the static portion (helix 3) of the coactivator binding pocket of ERα differs from than that of TIF-2.1.

### EXAMPLE 5

### LCoR is A Repressor of Ligand-Dependent Transcription Induced by Class I and Class II Nuclear Receptors

The effects of LCoR on transactivation by nuclear receptors were tested by transient transfection in COS-7 cells (FIG. 5), which revealed that LCoR is a repressor of ligand-dependent transcription of class I and II receptors. In FIGS. 5A, 5C, 5D, 5F, and 5H, LCoR represses ERα-, GR-, PR- and VDR-dependent transactivation. COS-7 cells were cotransfected with expression vectors for ERα HEG0 (A and C) or GR (D) or PR (F) or VDR (H), ERE3-TATA-pXP2 (A and C), GRE5/pXP2 (D and F) or VDRE3tk/pXP2 (H) luciferase reporter vectors, pCMV-β-gal as internal control, and LCoR/pSG5 or LSKAA/pSG5 expression vectors as indicated. Cells were treated with 10⁻⁷M of hormones (solid bars) or vehicle (open bars). Normalized luciferase activities (RLU) are the means±SEM from at least 3 experiments. The inset of FIG. 5A illustrates control western blot of ERα from extracts of COS-7 cells transfected with ERα HEG0 and 0, 500 or 1000 ng of LCoR/pSG5 in the absence or presence of estradiol. FIG. 5C illustrates that LCoR represses TIF-2 coactivation of ERα. Cells were transfected as in FIG. 5A with LCoR, TIF-2 or TIF2.1 as indicated. FIG. 5J illustrates a GAL4-LCoR fusion protein represses transactivation. COS-7 cells were transfected with 750 ng of 17 mer5tk/pXp2, with indicated amounts of GAL4-LCoR/pSG5 or 1000 ng of pSG5 or GAL4/pSG5. Normalized luciferase activities (RLU) are the means±SEM from at least 3 experiments. FIGS. 5B, 5E, 5G, 5I and 5K illustrate differing effects of HDAC inhibitor TSA on repression by LCoR. Transfections were performed as in the left-hand panels except that TSA (3 μM) was added.

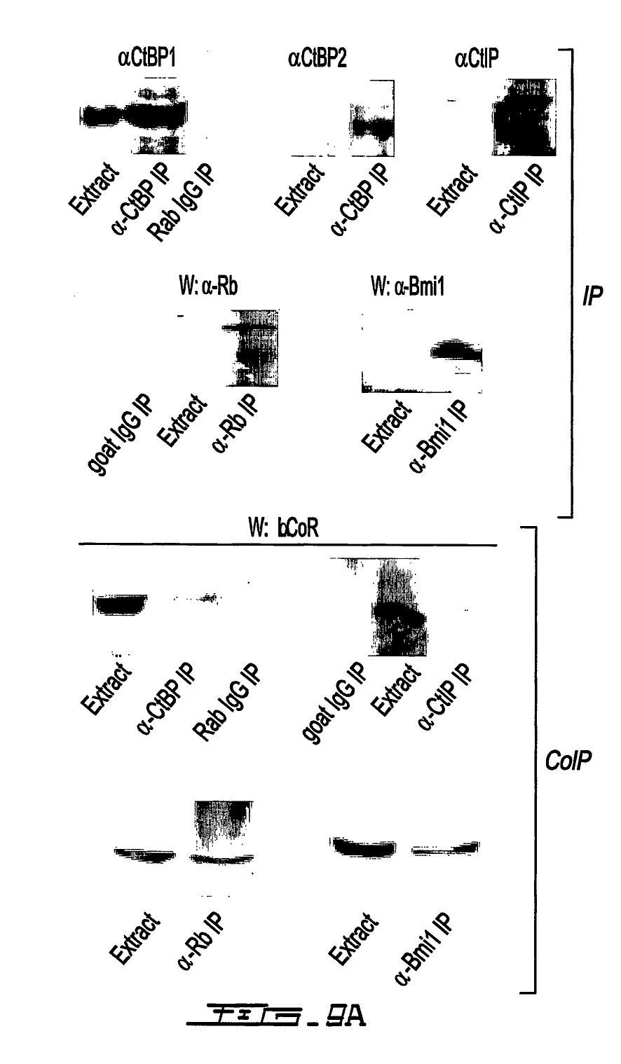

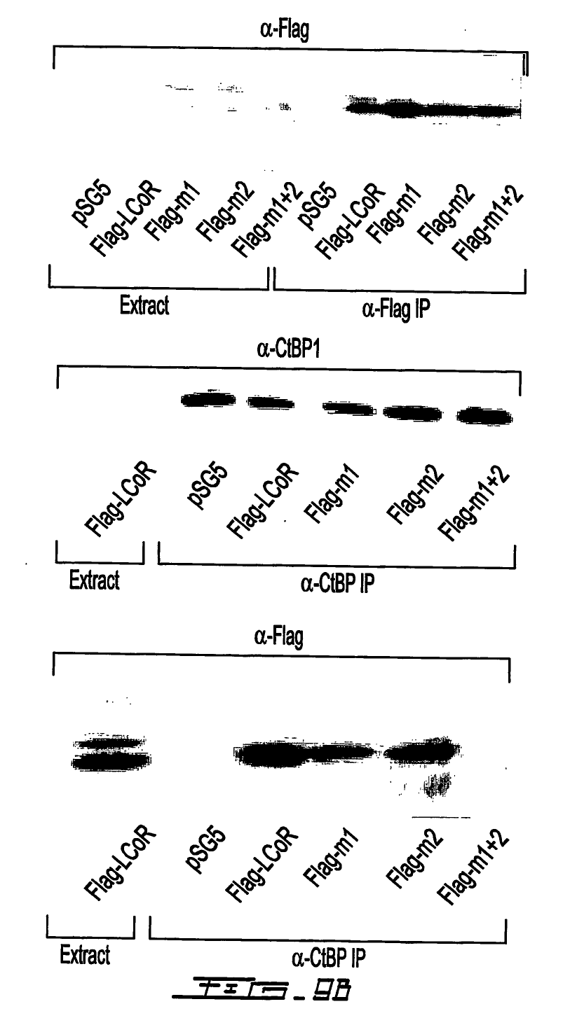

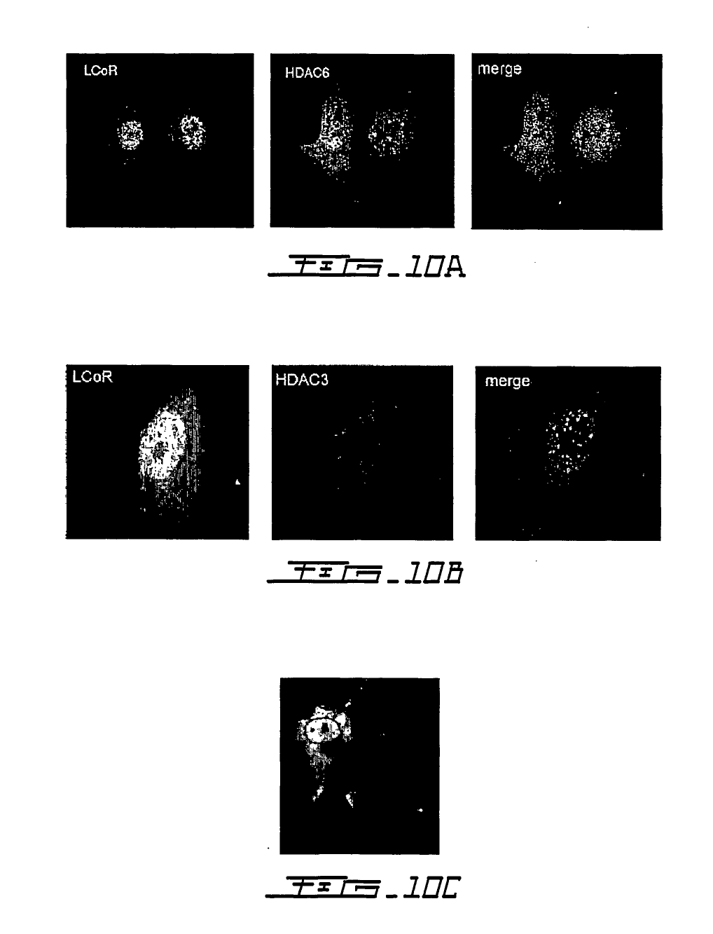

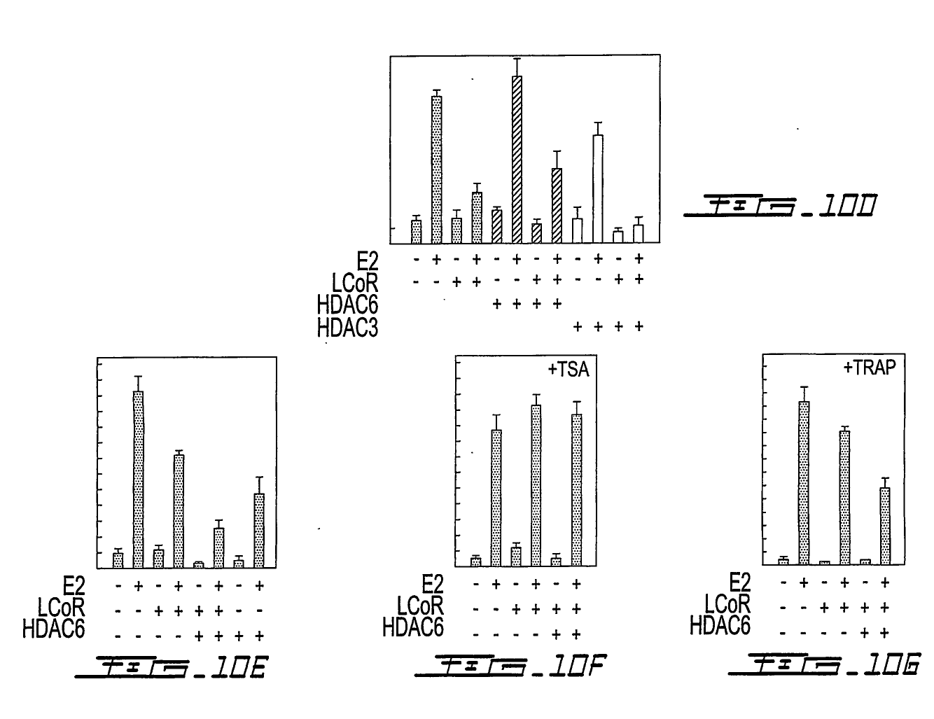

Coexpression of LCoR produced a dose-dependent repression of hormone-dependent transactivation by ERα, which was abolished by mutation of the NR box, as the LSKAA mutant had no effect on ERα function (FIG. 5A). Repression of estrogen-dependent gene expression was not due to downregulation of ERα protein in cells cotransfected with LCoR (FIG. 5A, inset). Similar results were obtained in MCF-7 and HEK293 cells. Consistent with LCoR and TIF-2 recognizing overlapping binding sites on ERα, LCoR repressed estrogen-dependent expression coactivated by TIF2 or TIF2.1 (FIG. 5C). Repressive effects of 1 μg of transfected LCoR on ligand-activated transcription on the order of 2.2-5-fold were observed in experiments with the glucocorticoid, progesterone and vitamin D receptors, (FIGS. 5D, F and H). In each case, mutation of the NR box disrupted transcriptional repression. Moreover, a GAL4-LCoR fusion repressed the activity of the 5×17 mer-tk promoter in a dose-dependent manner by 4-fold (FIG. 5J), whereas as free LCoR had no effect on the 5×17 mer-tk promoter. The mechanism of action of LCoR was investigated by analyzing the effect of the HDAC inhibitor trichostatin A (TSA) on repression of ligand-dependent transcription. Remarkably, while TSA completely abolished LCoR-dependent repression of ERα and GR function (FIGS. 7B and E), it had little or no effect on repression of PR or VDR function, or on repression by GAL-LCoR (FIGS. 5G, I and K), indicating that LCoR may function by HDAC-dependent and independent mechanisms.

### EXAMPLE 6

### LCoR Interacts Selectively With Histone Deacetylases

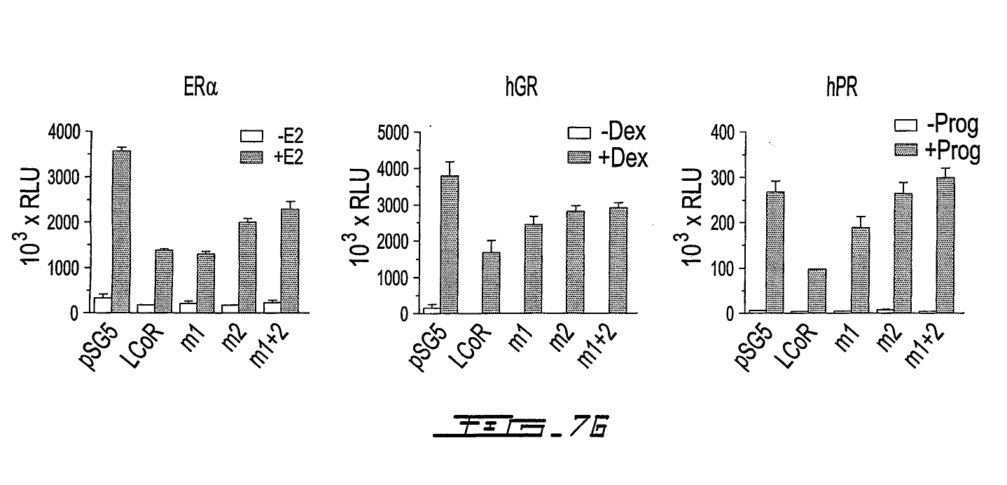

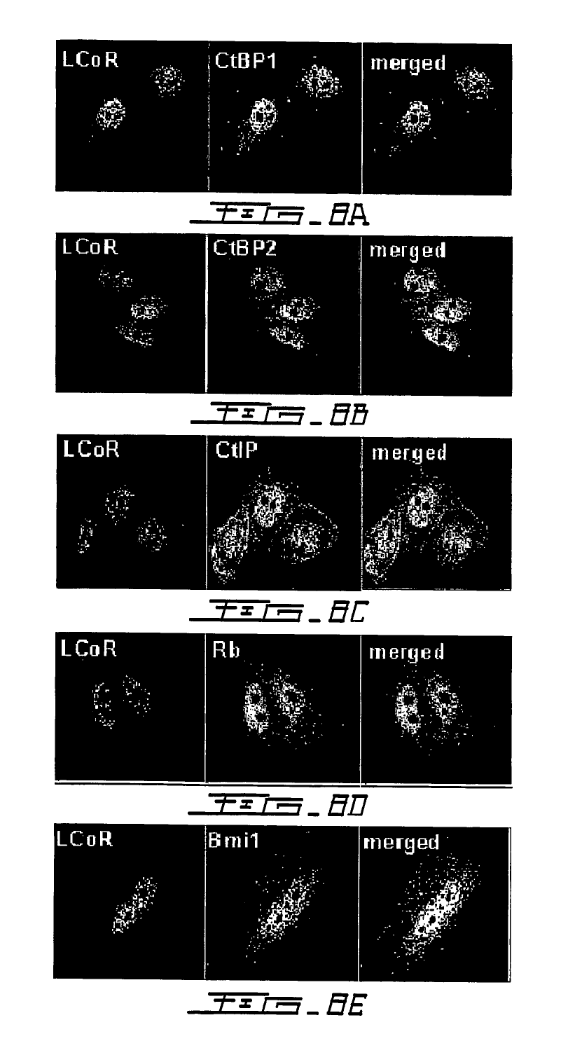

Pull-down assays performed with GST-LCoR and GST-LSKAA to screen for potential interactions with class I HDACs 1 and 3, and class II HDACs 4 and 6 revealed that both LCoR proteins interacted with HDACs 3 and 6, but not with HDACs 1 and 4 (FIG. 6A).

In FIG. 6A, HDACs 1, 3, 4, and 6 were in vitro translated and incubated with GST alone or with GST-LCoR or GST-LSKAA fusion proteins. The input (lane 1) represents 10% of the amount of labeled protein used in assays. FIG. 6B illustrates the association of tagged LCoR or LCoR-LSKAA with HDAC3. Lysates from COS-7 cells transiently transfected with HA-HDAC3 and Flag-LCoR or Flag-LSKAA, were precipitated with anti-Flag antibody. Cell extract and immunocomplexes were analyzed by Western blotting with anti-HDAC3 or anti-Flag. FIG. 6C illustrates endogenous LCoR coimmunoprecipitates with endogenous HDAC3. Immunoprecipitations from MCF-7 cell extracts were performed with either rabbit control IgG or anti-HDAC3 antibody, and immunoprecipitates were probed for HDAC3 or LCoR as indicated. FIG. 6D illustrates association of LCoR and LCoR-LSKAA with HDAC6. Lysates from COS-7 cells transiently cotransfected with HA-Flag-HDAC6 and HA-LCoR or HA-LSKAA, were precipitated with anti-Flag antibody and the immunocomplexes were analyzed by Western blotting with anti-HA or anti-Flag. FIG. 6E illustrates endogenous LCoR coimmunoprecipitates with endogenous HDAC6. Immunoprecipitations from MCF-7 cell extracts were performed with either rabbit control IgG or anti-HDAC6 antibody, and immunoprecipitates were probed for HDAC6 or LCoR as indicated.

Reciprocal coimmunoprecipitation experiments revealed an interaction between epitope-tagged LCoR or LCoR-LSKAA and HDAC3 (FIG. 6B). Moreover, interaction between endogenous LCoR and HDAC3 was confirmed by coimmunoprecipitation with an anti-HDAC3 antibody from extracts of MCF-7 cells (FIG. 6C). Identical results were obtained in extracts of HEK293 cells. Similarly, HA-LCoR and HA-LCoR-LSKAA were coimmunoprecipitated with HA-Flag-HDAC6 by an anti-Flag antibody (FIG. 6D), and endogenous LCoR coimmunoprecipitated with HDAC6 from extracts of MCF-7 cells (FIG. 6E). Taken together, these results indicate that LCoR can function to couple specific HDACs to ligand-activated nuclear receptors.

### EXAMPLE 7

### LCoR Interacts with C-Terminal Binding Protein (CtBP) Corepressors

FIGS. 7A-7G illustrates that LCoR interacts with C-terminal binding proteins. FIG. 7A is a schematic representation of LCoR showing CtBP binding sites 1 and 2, and the position of the Mfe1 site used to create C-terminally truncated LCoR. In FIG. 7B, GST pull-down assays were performed with in vitro translated CtBP1, and GST control (pGEX) or fusions with LCoR, LCoR-LSKAA or LCoR-Mfe1 deletion mutant. In FIG. 7C, GST pull-down assays were performed with in vitro translated CtBP1, and GST control (pGEX) or fusions with LCoR, LCoR-LSKAA or LCoR mutated in CtBP binding sites 1 (m1), 2 (m2) or 1 and 2 (m1+2). All GST fusion proteins were expressed at similar levels. FIG. 7D illustrates that LCoR coimmunoprecipitates with CtBPs. Extracts of MCF-7 cells were immunoprecipitated with rabbit control IgG or with a rabbit polyclonal anti-CtBP antibody, and immunoprecipitates were probed for CtBP1, CtBP2 or LCoR. FIGS. 7E and 7F illustrate colocalization of LCoR and CtBP1 (E) or CtBP2 (F) by confocal microscopy. In FIG. 7G, mutation of CtBP binding motifs attenuates repression by LCoR. COS-7 cells were cotransfected with expression vectors for ERα or GR or PR as indicated, along with ERE3-TATA-pXP2 or GRE5/pXP2 as appropriate, and either wild-type LCoR or LCoR mutated in CtBP binding motifs 1 or 2 as indicated.

Analysis of LCoR sequence (FIG. 7A) revealed PLDLTVR (a.a. 64) and VLDLSTK (a.a 82) motifs that are homologous to the PLDLS/TXR/K sequence defined as a binding site for the corepressor CtBP1. CtBP1, which was originally found as a protein that interacts with the C-terminus of E1A, functions by HDAC-dependent and -independent mechanisms (Chinnadurai, G. (2002) Mol. Cell, 9, 213-24), and is highly homologous to CtBP2. GST pull-down assays revealed an interaction between CtBP1 and wild-type LCoR, the LSKAA mutant, and an LCoR mutant lacking the C-terminal half of the protein (LCoR-Mfe1). CtBP1 binding was abolished only when both binding sites in LCoR were mutated (m1+2; FIG. 7C). While NADH can modulate CtBP function, no effect of NADH was seen on its interaction with LCoR in vitro.

CtBP1 and 2 are most efficiently immunoprecipitated with an antibody that recognizes both proteins. Western analysis suggested that the immunoprecipitates of MCF-7 cells contained mostly CtBP1 (FIG. 7D). Significantly, LCoR was coimmunoprecipitated with CtBP proteins under these conditions (FIG. 7D). A similar coimmunoprecipitation of LCoR was observed from extracts of HEK293 cells. In addition, immunocytochemical analysis of LCoR and CtBP1 expression in MCF-7 cells revealed a strongly overlapping expression pattern of the two proteins in discrete nuclear bodies (FIG. 7E). Similarly, the expression patterns of LCoR and CtBP2 overlapped in MCF-7 cell nuclei (FIG. 7F). Consistent with these findings, mutation of CtBP binding sites partially reduced the capacity of LCoR to repress ligand-dependent transcription by ERα and the GR (FIG. 7G), whereas mutation of site 2 or both sites largely abolished repression of PR-dependent transactivation. Taken together the above data shows that binding of CtBPs contributes to transcriptional repression by LCoR. Moreover, the greater dependence on the CtBP binding sites of LCoR for repression of progesterone-induced transactivation would be consistent with CtBP and its associated factors contributing to the TSA-insensitive repression of the PR observed above.

### EXAMPLE 8

### Nuclear Receptor Corepressor LCoR and Cofactor Histone Deacetylase 6 are Associated with Polycomb Group Transcriptional Repressor Complexes

We recently identified ligand-dependent corepressor LCoR as a coregulator of hormone-dependent transcription controlled by nuclear receptors. LCoR interacts with the corepressor C-terminal binding protein (CtBP) and histone deacetylases (HDACs) 3 and 6. While HDAC3 and LCoR are both nuclear proteins, the association of HDAC6 with LCoR is noteworthy as it is exclusively cytoplasmic in many cells. Here, we have analyzed the subcellular localization of LCoR and associated cofactors and their contribution to LCoR function. LCoR was distributed throughout the nucleus and was concentrated in nuclear bodies containing CtBP, CtBP-interacting protein CtIP, the retinoblastoma gene product (Rb), and BMI1, a component of polycomb group (PcG) transcriptional repressor complexes. In addition, endogenous LCoR coimmunoprecipitated with endogenous CtBP, CtIP, Rb, and BMI1, further establishing its association with PcG complexes. HDAC3 was distributed evenly throughout the nucleus and partially colocalized with LCoR. Remarkably, HDAC6 was partially nuclear in MCF-7 cells and colocalized with LCoR in PcG complexes. This colocalization was cell-specific, as HDAC6 remained fully cytoplasmic even when overexpressed with LCoR in COS-7 cells. Consistent with these findings, HDAC6 contributed to LCoR-dependent corepression of estrogen receptor □-dependent transcription in MCF-7 cells, but not in COS-7 cells, whereas HDAC3 enhanced LCoR corepression in COS-7 cells. Taken together these findings show that corepressor LCoR associates with PcG complexes, and that HDAC6 associates with these complexes in a cell-specific manner. Thus, HDAC6 functions cell-specifically as an LCoR cofactor and repressor of transcription.

Antibodies.

A rabbit polyclonal antipeptide antibody was raised against LCoR a.a 20-36 (QDPSQPNSTKNQSLPKA) fused to keyhole limpet hemocyanin, and purified over a peptide affinity column (Bethyl Laboratories, Montgomery Tex.). Rabbit polyclonal α-CtBP (sc-11390), goat polyclonal α-CtBP1 (sc-5963), goat polyclonal α-CtBP2 (sc-5967), goat polyclonal α-CtIP (sc-5970), goat polyclonal α-Rb (sc-1538), goat polyclonal α-Bmi1 (sc-8906), rabbit polyclonal α-Bmi1 (sc-10745), goat polyclonal HDAC3 (sc-8138), goat polyclonal HDAC6 (sc-5253), protein A-agarose and protein A+G-agarose were from Santa Cruz Biotechnology (Santa Cruz, Calif., USA). Cy3-donkey polyclonal α-goat (705-165-147) and Cy2-goat polyclonal α-rabbit (711-225-152), Cy3-donkey polyclonal α-rabbit (711-165-152), Cy2-donkey polyclonal α-mouse (715-225-150) were purchased from Jackson ImmunoResearch (West Grove, Pa., USA). Mouse monoclonal α-Flag M2 (F3165), and α-FLAG M2 HRP-conjugate (A-8592), monoclonal α-rabbit HRP conjugate (A2074), rabbit polyclonal α-goat HRP conjugate (A5420) were from Sigma (St. Louis, Mo.).

Recombinant Plasmids.

PSG5/LCoR, Flag-HDAC6/pcDNA3, HA-HDAC3/pCDNA3.1, Flag-LCoR/pcDNA3.1 and LCoR derivatives mutagenized in the CtBP binding motifs, PLDLTVR (LCoR a.a. 64-70; m1) and VLDLSTK (LCoR a.a. 82-88; m2) and the double mutant (m1+2) have been described (Renaud JP et al., 2000 Cell & Mol. Life Sci 57 1748-69.). LCoR cDNAs mutated in the CtBP binding motifs were subcloned downstream of Flag in pCDNA3.1.

Cell Culture and Transfections.

All cells were cultured under the recommended conditions. For immunocytochemistry, COS-7 cells grown on collagen IV-treated microscope slides in 6-well plates in DMEM, supplemented with 10% FBS were transfected in medium without serum with 12.5 μl of lipofectamine 2000 (Invitrogen, Burlington, Ont.) containing 1 μg each of pSG5/LCoR and HA-Flag-HDAC6/pcDNA3. Medium was replaced 24 h after transfection and cells were prepared for immunocytochemistry after 48 h as described below. For immunoprecipitation of tagged proteins, MCF-7 cells in 100 mm dishes were transfected with 10 μl of lipofectamine containing 10 μg of pSG5 vectors containing Flag-LCoR, Flag-m1, Flag-m2 or Flag-m1+2. For analysis of the effects of HDACs 3 or 6 on LCoR corepression, COS-7 cells (60-70% confluent) grown in DMEM without phenol red, supplemented with 10% FBS on 6-well plates were transfected in medium without serum with lipofectamine 2000 (Invitrogen, Burlington, Ontario, Canada) with 100 ng of ERα expression vectors as indicated, 300 ng of LCoR/pSG5, 300 ng of HA-HDAC3/pCDNA3.1 or Flag-LCoR/pcDNA3.1, 250 ng of ERE3-TATA-CAT reporter plasmid, 250 ng of internal control vector pCMV-βgal, and pBluescript carrier DNA to 4 μg. Medium was replaced 18 hr after transfection by a medium containing charcoal-stripped serum and ligand (10 nM) for 30 hr, as indicated. MCF-7 cells grown in 6-well plates were transfected similarly, except that cells were transfected at 90% confluence. MCF-7 cells were also grown in 24-well plates and were transfected using a ⅕^{th} scale. TSA and trapoxin were added to 500 nM and 50 nM, respectively, as indicated. Cells were harvested in 200 μl of reporter lysis buffer (Promega), and CAT assays were performed using an ELISA kit (Roche Diagnostics, Mannhein, Germany) according to the manufacturer's instructions. Note that the tranfection conditions above were chosen because the amounts of HDAC and LCoR expression vectors used led to selective repression of ERα-dependent transactivation without affecting expression of the β-galactosidase internal control.

Immunocytochemistry and Immunoprecipitations

Cells were cultivated on collagen IV-treated microscope slides in 6-well plates, fixed with 2% paraformaldehyde for 15 min at room temperature, washed (3×) with PBS, and permeabilized with 0.2% Triton X1001/5% BSA/10% horse serum in PBS. MCF-7 cells were then incubated with α-LCoR (1:500), and goat polyclonal antibodies against CtBP1, CtBP2, CtIP, Rb, HDAC3, HDAC6 or Bmi1 (1:50) in buffer B (0.2% Triton X100/5% BSA in PBS), for 1 h at room temperature. Cells were washed (3×) with PBS, and incubated with goat anti-rabbit-Cy2 and donkey anti-goat Cy3 (1:300) in buffer B for 1 h at room temperature. Transiently transfected COS-7 cells were incubated with α-LCoR (1:500), and anti-FLAG (1:300) to detect Flag-HDAC6. Cells were washed (3×) with PBS, and incubated with Cy3-donkey polyclonal a-rabbit (1:300), Cy2-donkey polyclonal α-mouse (1:400) in buffer B for 1 h at room temperature Slides were mounted with Immuno-Fluore Mounting Medium (ICN, Aurora, Ohio) and visualized using a Zeiss LSM 510 confocal microscope at 63× magnification.

For immunoprecipitation of endogenous CtBP, CtIP, Rb, or Bmi1, MCF-7 cells in 150 mm dishes were lysed 3 min at 4° C. in 1 ml of LB (150 mM NaCl/10 mM Tris-HCl pH 7.4/0.2 mM Na orthovanadate/1 mM EDTA/1 mM EGTA/1% Triton-100X/0.5% IGEPAL CA-630/protease inhibitor cocktail; Boehringer-Mannheim, Laval, Qc). Cell debris were pelleted by centrifugation (14,000 rpm, 5 min), and proteins immunoprecipitated with 4 μg of αCtBP or αCtIP or αRb or polyclonal rabbit αBMI1 or control rabbit or goat IgG at 4° C. overnight followed by 2 hours incubation at 4° C. with protein A agarose (for αCtBP, αBmi1, control rabbit IgG) or protein A+G agarose (for αCtIP or αRb or control goat IgG). Beads were washed (3×) with LB. Bound immunocomplexes were boiled in Laemmli buffer, separated by 10% SDS/PAGE, and blotted on PVDF membrane with α-LCoR ( 1/1000), α-CtBP1, α-CtBP2, α-CtIP, α-Rb or α-BMI1 (1:100), and detected by enhanced chemiluminescence (NEN Life Science Products, Boston, Mass.). For immunoprecipitation of tagged proteins, transfected MCF-7 cells were lysed 30 min at 4° C. in 1 ml of LB, 48 h after transfection. Supernatants were cleared, incubated overnight with 4 μg of aCtBP or α-Flag M2 antibody followed by 2 hours incubation with protein-A agarose or protein A+G agarose beads respectively. Beads were washed (3×) with LB and Western blotted as above. Dilutions of specific antibodies used for Western blotting were: α-CtBP1 , α-CtBP2 (1:100), α-Flag M2-peroxidase (1:100).

Association of LCoR with Polycomb Group Repressor Complexes

Our previous studies showed that LCoR interacts strongly and directly with CtBPs through tandem consensus motifs, and that the integrity of these motifs was essential for full corepression of hormone-dependent transcription. Colocalization of LCoR with CtBPs 1 and 2 in MCF-7 cell nuclei was confirmed by immunocytochemical analyses (FIGS. 8A and 8B). Both proteins were both broadly distributed in the nucleus and were also concentrated in discrete nuclear bodies. Given the functional interaction and the extensive overlap of CtBP and LCoR in the nucleus, we also investigated whether LCoR colocalized with CtBP-interacting proteins. CtBP-interacting protein (CtIP) was identified as a CtBP cofactor containing a PXLDLXXR motif, whose association with CtBP was disrupted by E1A. Subsequently, CtIP was found to interact directly with the retinoblastoma gene produc). Remarkably, similar to results obtained with CtBP, CtIP and LCoR showed strongly overlapping patterns of expression in discrete nuclear bodies (FIG. 8C). We also observed a substantial colocalization of LCoR and Rb (FIG. 8D).

Taken together, the above experiments strongly suggest that LCoR is associated with polycomb group (PcG) transcriptional repressor complexes. PcG proteins form large complexes containing several factors, visible as discrete nuclear structures. Distinct evolutionarily conserved complexes containing PcG components EED/EZH2 and BMI1/RING1 have been identified. Recent studies have linked CtBP1 and Rb to PcG complexes containing RING1 and BMI1. The presence of BMI1-containing PcG complexes was probed with an antibody against BMI1 (FIG. 8E), which revealed nuclear structures similar to those described in FIGS. 8A-D, and a strong colocalization with LCoR.

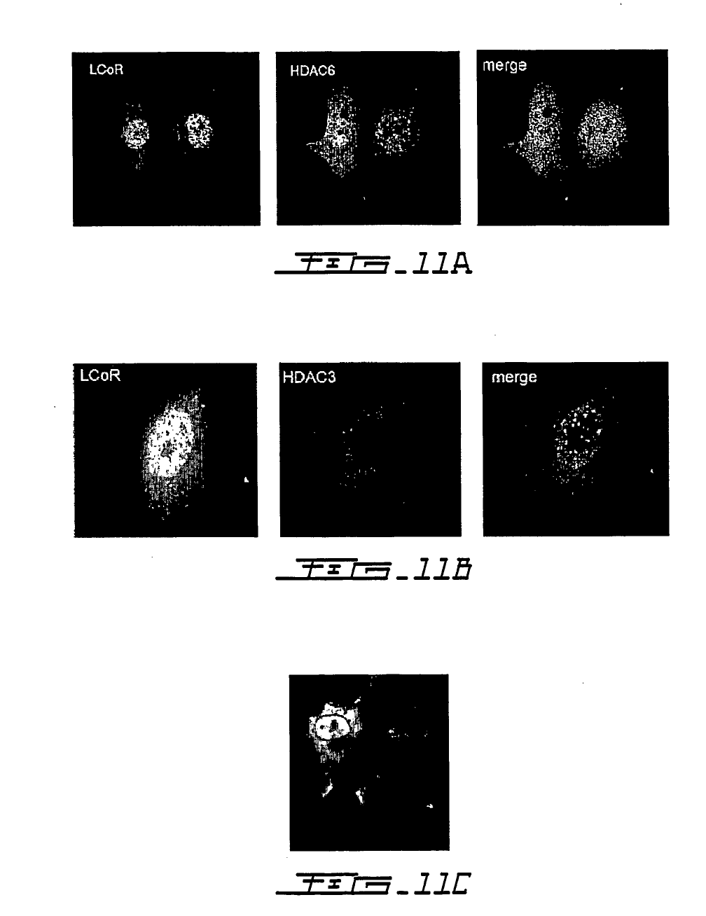

The association of LCoR with PcG complexes and associated proteins was further supported by coimmunoprecipitation experiments from MCF-7 cell extracts in which endogenous LCoR was detected in immunoprecipitates of endogenous proteins generated with antibodies directed against CtBP, CtIP, Rb and BMI1, but not with control antibody (FIG. 9). The coimmunoprecipitation of CtIP, and by extension Rb, and LCoR is remarkable given that CtIP and LCoR interact with CtBP through common PXLDLXXR motifs. While repressors such as the Kruppel zinc finger protein Ikaros can interact simultaneously with CtBP and CtIP, no evidence was found for LCoR binding directly to CtIP or Rb in vitro in GST pull-down experiments, indicating that their association in vivo is indirect. Moreover, tagged wild-type LCoR or LCoR mutated in one of its two CtBP binding sites coimmunoprecipitated with endogenous CtBPs from extracts of MCF-7 cells, whereas no coimmunoprecipitation was observed in cells expressing an LCoR derivative (m1+2) mutated in both sites (FIG. 3, bottom panel). This is consistent with the observation that mutation of both CtBP binding sites of LCoR was required to abolish its interaction with CtBP in vitro (13). While the results show that LCoR binds directly to CtBPs through its cognate binding motifs in vivo, they also indicate that the two proteins do not also associate indirectly through stable interaction of LCoR with other components of PcG complexes.

HDAC6 is Associated with LCoR in PcG Complexes

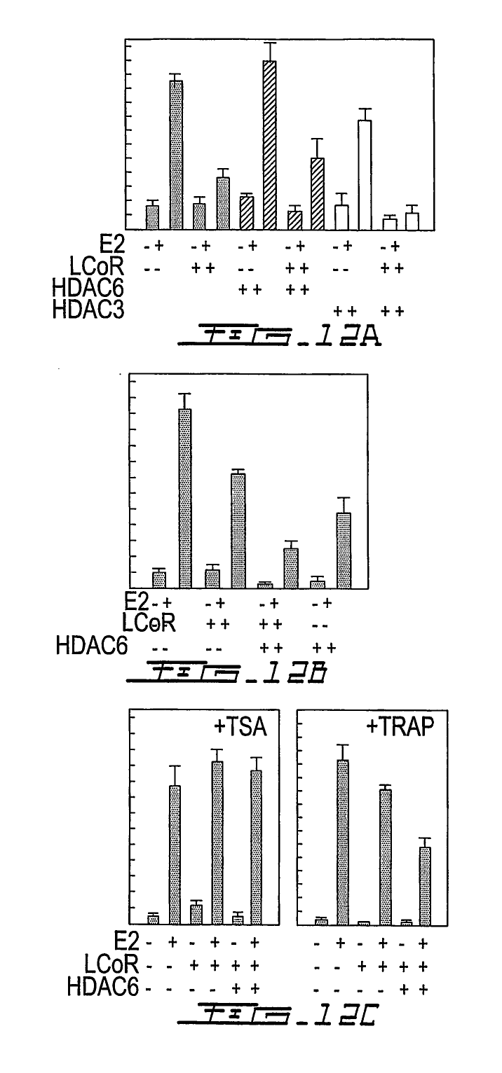

We were interested in examining the function of HDACs 3 and 6 as cofactors of LCoR and there association with LCoR in vivo. Our previous studies showed that HDACs 3 and 6 interacted with LCoR in vitro, and, importantly, that endogenous LCoR coimmunoprecipitated with endogenous HDACs 3 and 6 from MCF-7 cell extracts. HDAC6 is largely cytoplasmic in most cells due to the presence of a potent nuclear export signal at the N-terminus of the protein. However, the protein can become partially nuclear in B16 melanoma cells induced to differentiate, suggesting that it may regulate gene expression under some conditions. Strikingly, we found that HDAC6 is partially nuclear in MCF-7 cells, and, moreover, showed strong colocalization with LCoR in PcG complexes (FIG. 11A). The subcellular distribution of HDAC6 differs from that of HDAC3, which was detected more evenly through the nucleus and in a pattern partially overlapping with that of LCoR (FIG. 4B). These findings are consistent with other studies showing that HDAC3 is nuclear or partially nuclear in many cell types. The association of HDAC6 with nuclear LCoR is clearly cell-specific, as we found that it remained entirely cytoplasmic in COS-7 cells even when overexpressed along with LCoR by transient transfection (FIG. 1C).

Cell-Specific Repression of Hormone-Dependent Transactivation by HDAC6

Cotransfection experiments showed that the cell-specific colocalization of HDAC6 was consistent with it capacity to promote LCoR-dependent corepression. Cotransfection of HDAC6 in COS-7 cells had no effect on LCoR-dependent corepression of hormone-dependent transactivation by ERα (FIG. 12A). As a control for repressive effects of HDAC cotransfection in COS-7, we performed a similar experiment with HDAC3, which repressed transcription on its own and enhanced transcriptional repression by LCoR (FIG. 12A). In contrast to the results obtained in COS-7 cells, HDAC6 partially repressed ERα-dependent transactivation in MCF-7 cells, and enhanced corepression by LCoR (FIG. 12B). Note that the transfections in FIG. 12B were performed with limiting amounts of LCoR and HDAC6, under conditions which repressed estrogen-dependent reporter gene activity, without affecting the internal control plasmid. Importantly, effects of HDAC6 were abolished by the HDAC inhibitor trichostatin A, but not by the inhibitor trapoxin (FIGS. 12D and 12E), to which HDAC6 is resistant. Taken together, these results show that LCoR is associated with polycomb group transcriptional repressor complexes in vivo and support a role for HDAC6 as a cell-specific LCoR cofactor. Moreover, they indicate that HDAC6 functions as a repressor of transcription in cells in which it is nuclear.

Although preferred embodiments of the invention have been described herein, it will be understood by those skilled in the art that variations may be made thereto without departing from the spirit of the invention or the scope of the appended claims.

1. An isolated corepressor polypeptide encoded by the nucleotide sequence as set forth in FIG. 1D and having an amino acid sequence which comprises at least one LXXLL nuclear receptor interacting NR box motif wherein L is leucine and X is any amino acid residue, said polypeptide operably interactable with a nuclear receptor to actively repress transcription of DNA.

2. The isolated polypeptide of claim 1, wherein said polypeptide is operably interactable with a nuclear receptor in one of a ligand-dependent and partially ligand-dependent manner.

3. The isolated polypeptide of claim 2, wherein the nuclear receptor comprises a class I or a class II nuclear receptor.

4. The isolated polypeptide of claim 3, wherein the nuclear receptor is selected form the group consisting of ERα, ERβ, GR, PR, VDR, RARα, RARβ, RARγ and RXRα.

5. An isolated corepressor polypeptide essentially having an amino acid sequence as set forth at FIG. 1D comprising at least one modification of the amino acid sequence.

6. The isolated polypeptide of claim 5, wherein said modification comprises at least one point mutation in the region of the sequence from nucleotides 53 to 57.

7. The isolated polypeptide of claim 6, wherein the sequence from nucleotides 53 to 57 comprises the sequence LSKAA.

8. An isolated corepressor polypeptide encoded by the nucleotide sequence as set forth in FIG. 1D and having within its amino acid sequence at least two C-terminal binding protein interaction motifs, said first C-terminal binding protein interaction motif comprising the sequence PLDLTVR, and said second C-terminal binding protein interaction motif comprising the sequence VLDLSTK, said polypeptide operably interactable with a C-terminal binding protein (CtBP) corepressor in a pathway to repress expression of DNA.

9. The isolated polypeptide of claim 8, wherein the CtBP corepressor is selected from the group consisting of CtBP1 and CtBP2.

10. The isolated polypeptide of claim 8 comprising the amino acid sequence as set forth in FIG. 1D.

11. An isolated polynucleotide coding for the polypeptide of claim 5.

12. An expression vector comprising the polynucleotide of claim 11 operably linked to a promoter for expression in a host cell.

13. A host cell stably transformed with the expression vector of claim 12.

14. An antibody that specifically binds to the polypeptide of claim 1.

15. An antibody that specifically binds to the polypeptide of claim 5.

16. An antibody that specifically binds to the polypeptide of claim 8.

17. A transgenic knock-out mouse comprising disruption in an endogenous gene which encodes for a corepressor polypeptide having a sequence as set forth in FIG. 1D, wherein said disruption has been introduced into its genome by a recombinant DNA construct stably integrated into the genome of said mouse or an ancestor thereof, wherein the disruption of the corepressor gene reduces expression of said corepressor causing altered active transcription of DNA associated with the corepressor.

18. The transgenic knock-out mouse of claim 17, wherein the altered active transcription of DNA is increased relative to wild type.

19. A method of modulating a cell comprising a gene which encodes for a corepressor polypeptide having a sequence as set forth in FIG. 1D, said method comprising the steps of introducing into said cell the isolated polynucleotide according to claim 5, whereby expression of the corepressor polypeptide is modulated.

20. A method of inhibiting ligand-dependent transactivation in a cell by one of a class I and class II nuclear receptor comprising subjecting said cell to a corepressor amount of the polypeptide of claim 1.

21. The method of claim 20, wherein the nuclear receptor is selected from the group consisting of ERα, ERβ, GR, PR, VDR, RARα, RARβ, and RARγ.

22. A method of repressing nuclear-receptor mediated transcription in a cell comprising providing a ligand-dependent corepressor amount of the polypeptide of claim 1 to said cell.

23. A method of modulating steroid hormone signaling in a cell comprising providing a ligand-dependent corepressor amount of the polypeptide of claim 1 to said cell.

24. A method of regulating gene expression in a cell comprising providing the polypeptide as set forth at claim 8, wherein the polypeptide is operable to interact with at least one protein in a pathway to regulate gene expression.

25. The method of claim 24, wherein the protein comprises a C-terminal binding protein corepressor.

26. The method of claim 25 wherein the C-terminal binding protein corepressor is selected from the group consisting of CtBP-1 and CtBP-2.

27-33. (canceled)

34. A method for assaying for compounds capable of modulating the activity of a corepressor polypeptide of claim 1 or an active variant thereof to actively modify transcription of DNA comprising the steps of:
(a) providing a corepressor polypeptide of claim 1 or an active variant thereof;
(b) contacting the corepressor polypeptide with a nuclear receptor in the presence and absence of the compound; and
(c) measuring the modulation in activity of repression of DNA translation of the corepressor polypeptide.

35. A method for assaying for compounds capable of affording selective recruitment of the corepressor polypeptide of claim 1 in the presence of a ligand of a nuclear receptor, wherein the corepressor is operably interactable with the nuclear receptor to actively repress transcription of DNA in the presence of the ligand.

36. The method of claim 35, wherein the ligand comprises estrogen or an estrogen-like compound and the repressed DNA transcription products are implicated in hormone-dependent cancer.

37. The method of claim 36, wherein the hormone-dependent cancer is selected from the group consisting of hormone-dependent breast cancer and hormone-dependent uterine cancer.
